(19) United States
(12) Patent Application Publication
(10) Pub. No.: US 2025/0235416 A1
(43) Pub. Date: Jul. 24, 2025

(54) METHOD FOR PREPARATION OF N-ACETYL CYSTEINE AMIDE, DERIVATIVES AND TABLETS THEREOF

(71) Applicant: NACUITY PHARMACEUTICALS, INC., Fort Worth, TX (US)

(72) Inventors: G. Michael Wall, Fort Worth, TX (US); Gerald D. Cagle, Fort Worth, TX (US)

(21) Appl. No.: 19/173,475

(22) Filed: Apr. 8, 2025

Related U.S. Application Data
(63) Continuation-in-part of application No. 18/770,850, filed on Jul. 12, 2024.
Application No. 18/770,850 is a continuation-in-part of application No. 18/678,550, filed on May 30, 2024.
Application No. 18/678,550 is a continuation-in-part of application No. 18/094,459, filed on Jan. 9, 2023.
Application No. 18/094,459 is a continuation-in-part of application No. 17/366,913, filed on Jul. 2, 2021, now Pat. No. 11,548,851.
Application No. 17/366,913 is a continuation-in-part of application No. 16/818,416, filed on Mar. 13, 2020, now Pat. No. 11,091,433.
Application No. 16/818,416 is a continuation-in-part of application No. 16/137,262, filed on Sep. 20, 2018, now Pat. No. 10,590,073.
(60) Provisional application No. 62/561,129, filed on Sep. 20, 2017.

(51) Int. Cl. A61K 31/164; A61K 9/20

(52) U.S. Cl. CPC A61K 31/164; A61K 9/2013; A61K 9/2018; A61K 9/2054; A61K 9/2095

(57) ABSTRACT

Provided herein are tablet(s) comprising N-acetylcysteine amide (NACA) and a NACA-acceptable, biocompatible excipient and optionally one or more pharmaceutically acceptable additives, binders, or fillers. Also provided are methods for making the tablets comprising N-acetylcysteine amide (NACA) and a NACA-acceptable, biocompatible excipient and optionally one or more pharmaceutically acceptable additives, binders, or fillers, e.g., comprising slugged tablets, wherein the slugged tablets have greater than 90, 91, 92, 93, 94, 95, 96, 97, 98, or 100% of the slugged tablets meet specifications; target width of 10 to 12 mm, or 11 mm; target weight of approximately 475 to 525 mg; and thickness range of approximately 4 to 6 mm, 4.5 to 5.5 mm or 5 mm. Pharmaceutical composition(s) in the form an oral tablet or capsule comprising N-acetylcysteine amide (NACA) for administration of the pharmaceutical composition to a human.

### CROSS-REFERENCE TO RELATED APPLICATIONS

This application is a Continuation-in-Part of U.S. Ser. No. 18/770,850, filed Jul. 12, 2024, entitled “Method for Preparation of N-Acetyl Cysteine Amide or DI-N-Acetyl Cystineamide and Derivatives”, which is a Continuation-in-Part of U.S. Ser. No. 18/678,550, filed May 30, 2024, entitled “Method for Preparation of N-Acetyl Cysteine Amide or DI-N-Acetyl Cystineamide and Derivatives”, which is a Continuation-in-Part of U.S. Ser. No. 18/094,459, filed Jan. 9, 2023, entitled “Method for Preparation of N-Acetyl Cysteine Amide or DI-N-Acetyl Cystineamide and Derivatives Thereof”, which is a Continuation-in-Part of U.S. Ser. No. 17/366,913, filed Jul. 2, 2021, entitled “Method for Preparation of N-Acetyl Cysteine Amide and Derivatives Thereof”, now U.S. Pat. No. 11,548,851, which is a Continuation-in-Part of U.S. Ser. No. 16/818,416, filed Mar. 13, 2020, entitled “Method for Preparation of N-Acetyl Cysteine Amide and Derivatives Thereof”, now U.S. Pat. No. 11,091,433, which is a Continuation-in-Part of U.S. Ser. No. 16/137,262, filed Sep. 20, 2018, entitled “Method for Preparation of N-Acetyl Cysteine Amide and Derivatives Thereof”, now U.S. Pat. No. 10,590,073, which claims priority to U.S. Provisional Application Ser. No. 62/561,129, filed Sep. 20, 2017, entitled “Method for Preparation of N-Acetyl Cysteine Amide and Derivatives Thereof”, the entire contents of each of which are incorporated herein by reference.

### STATEMENT OF FEDERALLY FUNDED RESEARCH

None.

### TECHNICAL FIELD

The present disclosure relates in general to the field of methods for preparing N-Acetyl Cysteine Amide (N-acetylcysteine amide, NACA), (2R,2R′)-3,3′-disulfanediyl bis(2-acetamidopropanamide, diNACA), and derivatives thereof.

### BACKGROUND

Without limiting the scope of the disclosure, its background is described in connection with synthesis of N-Acetyl Cysteine Amide and diNACA.

One such method is taught in U.S. Patent Publication No. 20170183302, filed by Warner, et al., entitled “Method for Preparation of N-Acetyl Cysteine Amide”. Briefly, these inventors teach a process for the preparation of N-acetyl-L-cysteine amide (NACA) starting with N-acetyl-L-cysteine that involves contacting N-acetyl-L-cysteine with an organic alcohol and an inorganic acid to form an organic solution containing N-acetyl-L-cysteine ester; neutralizing the acid in the organic solution with an aqueous solution containing a base to form a neutralized mixture; separating an organic solution containing N-acetyl-L-cysteine ester from the neutralized mixture; removing the N-acetyl-L-cysteine ester from the organic solution under reduced pressure; and contacting the N-acetyl-L-cysteine ester with ammonia.

Another such method for the preparation of N-acetyl cysteine amide (NACA) was previously described by Martin, et al., entitled “Amides of N-Acylcysteines as Mucolytic Agents”, J. Med. Chem. 1967, 10, 1172-1176.

However, a need remains for developing an efficient method for the effective, large-scale synthesis of N-acetyl cysteine amide that provides the product in high chemical yields and, in particular, high chemical and enantiomeric purity, without the need for chromatography.

### SUMMARY

As embodied and broadly described herein, an aspect of the present disclosure relates to a tablet comprising N-acetylcysteine amide (NACA) and a NACA-acceptable, biocompatible excipient and optionally one or more pharmaceutically acceptable additives, binders, or fillers, also referred to as a NACA tablet. In one aspect, the one or more pharmaceutically acceptable additives, binders, or fillers are selected from microcrystalline cellulose, lactose monohydrate, croscarmellose sodium and stearic acid. In another aspect, the NACA comprises from 1, 2, 3, 4, 5, 6, 7, 8, 9, 10, 15, 20, 25, 30, 35, 40, 45, 50, 55, 60, 70 to 75 weight percent (wt. %) of the tablet. In another aspect, the NACA comprises 10, 20, 30, 40, 50, 60, or 70 wt. % of the tablet. In another aspect, the tablet is 100, 200, 250, 300, 350, 400, 450, 500, 600, 700, 750, 800, 900, 1,000, 1,250, 1,500, 1,750, or 2,000 mg. In another aspect, the lactose is 8.575 wt. %, but can range from 0, 1, 2, 3, 4, 5, 6, 7, 8, 9 10, 15, 20, 25, 30, 35, 40, 45, 50, 55, 60 to 66 wt. % with corresponding adjustments to other components. In another aspect, the microcrystalline cellulose is 34.425 wt. %, but can range from 0, 1, 2, 3, 4, 5, 6, 7, 8, 9 10, 15, 20, 25, 30, 35, 40, 45, 50, 55, 60, 70, 75, 80, 85, 90 to 91 wt. % with corresponding adjustments to other components. In another aspect, the croscarmellose sodium is 5.0 wt. %, but can range from 0, 1, 2, 3, 4, 5, 6, 7, 8 to 9 wt. % with corresponding adjustments to other components. In another aspect, the stearic acid is 1.0 wt. %, but can range from 0.1, 0.2, 0.3, 0.4, 0.5, 0.6, 0.7, 0.8, 0.9, 1, 2, 3, 4, 5 to 5.6 wt. % with corresponding adjustments to other components. In another aspect, the tablets are dry granulated by roller compaction. In another aspect, the tablets are dry granulated by slugging. In another aspect, the NACA tablet weight is in the range of approximately 475 to 525 mg. In another aspect, the NACA tablet thickness is in the range of approximately 4 to 6 mm, 4.5 to 5.5 mm, or about 5 mm. In another aspect, the NACA tablet width is in the range of approximately 10 to 12 mm, or about 11 mm. In another aspect, the tablet hardness is in the range of approximately 3 to 12 kp, or 6 kp. In another aspect, the NACA tablet disintegration time is less than 20, 15, 10 or 5 minutes or approximately less than 1 minute. In another aspect, the NACA tablet friability is not more than 5, 4, 3, 2, or 1.0%, or approximately less than 1%. In another aspect, the NACA tablet tablets content uniformity complies to meets or exceeds US Pharmacopeia (USP) 905. In another aspect, the NACA tablet is a white to off-white, round, biconvex tablet. In another aspect, the NACA tablet is a coated or colored tablet. In another aspect, the NACA tablet has an elliptical or oval shape.

As embodied and broadly described herein, an aspect of the present disclosure relates to a method of making a pharmaceutical composition in the form an oral tablet comprising N-acetylcysteine amide (NACA), comprising: compressing the NACA and the NACA-acceptable, biocompatible excipient to form first tablets, crushing or milling the first tablets, and compressing the crushed or milled first tablets into slugged tablets, wherein the slugged tablets have greater than 90, 91, 92, 93, 94, 95, 96, 97, 98, or 100% of the slugged tablets meet specification, wherein the composition has a pharmacokinetic (PK) profile comprising mean plasma concentrations of NACA and NAC ranging from about 200 to 1200 ng/ml after administration of the pharmaceutical composition to a human, also referred to as a NACA tablet. In one aspect, the slugging the tablet comprises compressing the crushed or milled first tablets into slugged tablets, wherein the slugged tablets have greater than 90, 91, 92, 93, 94, 95, 96, 97, 98, or 100% of the slugged tablets meet specification. In another aspect, the tablet hardness is in the range of approximately 3 to 12 kp, or 6 kp. In another aspect, the NACA tablet disintegration time is less than 20, 15, 10 or 5 minutes or approximately less than 1 minute. In another aspect, the NACA tablet friability is not more than 5, 4, 3, 2, or 1.0%, or approximately less than 1%. In another aspect, the NACA tablet tablets content uniformity complies to meets or exceeds US Pharmacopeia (USP) 905. In another aspect, the NACA tablet is a white to off-white, round, biconvex tablet. In another aspect, the NACA tablet is a coated or colored tablet. In another aspect, the NACA tablet has an elliptical or oval shape.

As embodied and broadly described herein, an aspect of the present disclosure relates to a capsule comprising N-acetylcysteine amide (NACA) and a NACA-acceptable, biocompatible excipient and optionally one or more pharmaceutically acceptable additives, binders, or fillers.

This disclosure describes an efficient method or process for the preparation of NACA and diNACA in high chemical yields and high enantiomeric purity. Dimethyl 3,3′-disulfanediyl(2R,2′R)-bis(2-aminopropanoate) dihydrochloride is commonly referred to herein as L-cystine dimethyl ester dihydrochloride. Dimethyl 3,3′-disulfanediyl(2R,2′R)-bis(2-acetamidopropanoate) is commonly referred to herein as di-N-acetylcystine dimethyl ester or Di-NACMe. (2R,2′R)-3,3′-disulfanediylbis(2-acetamidopropanamide) is commonly referred to herein as diNACA. (R)-2-acetamido-3-mercaptopropanamide is commonly referred to herein as NACA or NPI-001 or N-acetylcysteine amide. Specifically, disclosed herein is a process comprising: contacting cystine with an alcohol and a chlorinating reagent to form an organic solution containing L-cystine dimethylester dihydrochloride; combining dried or undried L-cystine dimethylester dihydrochloride with a triethylamine, an acetic anhydride, and an acetonitrile to form a di-N-acetylcystine dimethylester; mixing dried di-N-acetylcystine dimethylester with ammonium hydroxide to form a di-N-acetylcystine amide; and reducing dried di-N-acetylcystine dimethylester into N-acetylcysteine amide with dithiothreitol, triethylamine and an alcohol. In one aspect, the alcohol is an organic alcohol selected from an alkyl alcohol, methanol, ethanol, propanol, iso-propanol or butanol. In another aspect, the step of contacting L-cystine with an alcohol and a chlorinating reagent to form an organic solution containing L-cystine dimethylester dihydrochloride is conducted at −10 to 10° C. and then heated to reflux at 65 to 70° C. to completion. In another aspect, the chlorinating agent is thionyl chloride. In another aspect, there is a solvent exchange between the contacting and the combining steps. In another aspect, the step of combining is performed at −10 to 10° C. In another aspect, a precipitate formed in the combining step that is filtered and washed with ethyl acetate before drying under vacuum. In another aspect, the step of combining uses at least 15 volumes acetonitrile at −10 to 10° C. before adding at least 4 equivalents of triethylamine followed by at least 2 equivalents of acetic anhydride. In another aspect, the organic solvent is ethyl acetate. In another aspect, the process further comprises drying the organic solution removed from the neutralized mixture with a drying agent. In another aspect, the process further comprises the step of free-basing the triethylamine from the di-N-acetylcystine dimethylester with saturated sodium bicarbonate after reaction completion. In another aspect, the ammonia is provided in the form of aqueous ammonium hydroxide. In another aspect, the contacting of the N-acetyl-L-cystine ester with ammonia is performed at 0° C. In another aspect, no metals are used in the reduction of di-N-acetylcystine dimethylester into N-acetylcysteine amide. In another aspect, the step of removing the organics under reduced pressure is performed at about 45° C. or less. In another aspect, the step of removing the organics under reduced pressure is performed at about 35° C. or less. In another aspect, the step of removing the organics under reduced pressure is performed at about 30° C. or less. In another aspect, the step of removing the organics under reduced pressure is performed at about 45° C. In another aspect, the organic solution removed from the neutralized mixture is filtered to remove solids. In another aspect, the one or more reducing agents is selected from triphenylphosphine, thioglycolic acid or dithiothreitol and the organic solvent is tetrahydrofuran (THF), dichloromethane (DCM), isopropanol (2-propanol), or ethanol.

In another embodiment, the present disclosure includes a compound having a formula:

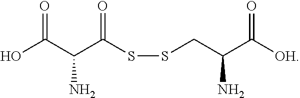 [SMILES: N[C@H](C(=O)O)C(=O)SSC[C@H](N)C(=O)O]

In another embodiment, the present disclosure includes a compound having a formula:

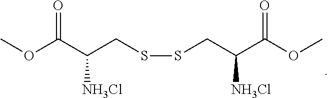

In another embodiment, the present disclosure includes a compound having a formula:

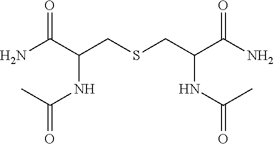 [SMILES: CC(=O)NC(CSCC(NC(C)=O)C(N)=O)C(N)=O]

In yet another embodiment, the present disclosure includes a process for making N-acetylcysteine amide comprising:

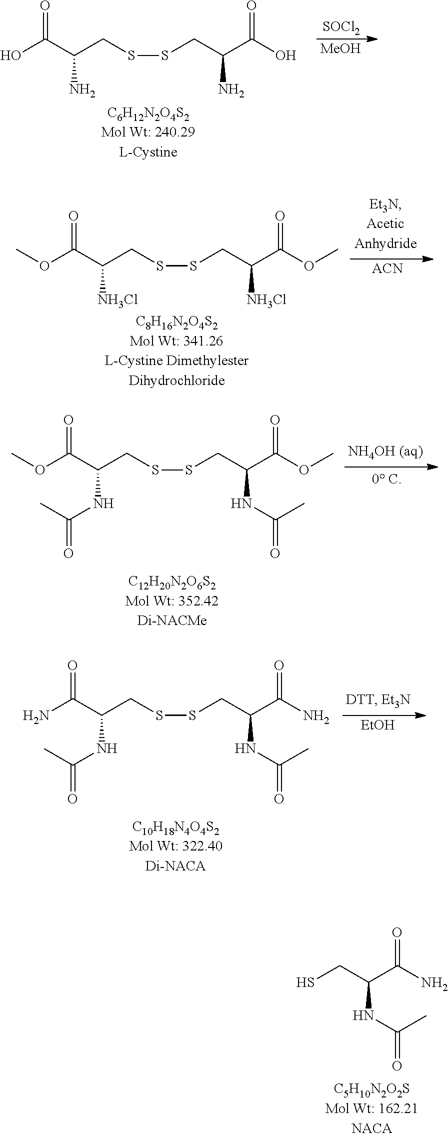

In another embodiment, the present disclosure includes a process for making N-acetylcysteine amide comprising: contacting L-cystine with an alcohol and a chlorinating reagent to form an organic solution containing L-cystine dimethylester dihydrochloride; combining dried L-cystine dimethylester dihydrochloride with a triethylamine, an acetic anhydride, and an acetonitrile to form a di-N-acetylcystine dimethylester; mixing dried di-N-acetylcystine dimethylester with ammonium hydroxide to form a di-N-acetylcystine amide; and reducing dried di-N-acetylcystine amide into N-acetylcysteine amide with dithiothreitol, triethylamine, and an alcohol, wherein the reduction is without the presence of a metal. In one aspect, the one or more reducing agents is selected from tris (2-carboxyethyl)phosphine, thioglycolic acid, dithiothreitol, and the organic solvent is THF or dichloromethane (DCM), isopropanol, or ethanol, and the base is triethylamine and or sodium bicarbonate.

In another embodiment, the present disclosure includes a process for making (2R,2R′)-3,3′-disulfanediyl bis(2-acetamidopropanamide) (DiNACA) comprising: contacting cystine with an alcohol and a chlorinating reagent to form an organic solution containing L-cystine dimethylester dihydrochloride; combining dried or undried L-cystine dimethylester dihydrochloride with a triethylamine, an acetic anhydride, and an acetonitrile to form a di-N-acetylcystine dimethylester; and mixing dried di-N-acetylcystine dimethylester with ammonium hydroxide to form a di-N-acetylcystine amide.

### BRIEF DESCRIPTION OF THE DRAWINGS

For a more complete understanding of the features and advantages of the present disclosure, reference is now made to the detailed description of the disclosure along with the accompanying figures and in which:

FIG. 1 shows a basic chemical synthesis of the present disclosure.

FIG. 2A shows a view of NPI-001 (NACA) molecule ‘A’ without atom labelling. All non-hydrogen atoms are shown with thermal ellipsoids set at the 50% probability level. Color code: Carbon, grey; H, white; O, red; S, yellow.

FIG. 2B shows a portion of NACA derived from starting material L-Cystine.

FIG. 3 shows a view of a unit cell an axis of NPI-001 containing complete molecules. All atoms are shown with thermal ellipsoids set at the 50% probability level. Color code: Carbon, grey; H, white; O, red; S, yellow.

FIG. 4 shows results of liquid chromatography with mass spectrometric detection.

FIG. 5 shows Simulated (120 K) XRPD 2θ diffractogram of NPI-001.

FIG. 6 shows ¹H-NMR of NACA.

FIG. 7 shows ¹H-NMR assignments for NACA (¹H and ¹³C assignments are based on analysis of the 1D ¹H NMR, ¹H-¹³C HSQC and ¹H-¹³C HMBC spectra.),

FIG. 8 shows ¹³C-NMR of NACA.

FIG. 9 shows ¹³C-NMR assignments for NACA. (¹H and ¹³C assignments are based on analysis of the 1D ¹H NMR, ¹H-¹³C HSQC and ¹H-¹³C HMBC spectra).

FIG. 10 shows combined thermogravimetry/differential scanning calorimetry scans of NACA,

FIG. 11 shows Fourier transform-infrared (FT-IR) spectrum of NACA.

FIG. 12 is a representative Method-I Chromatogram showing NACA, Intermediates and Starting Material.

FIG. 13 is a representative Chromatogram of Method-II Showing B1 and B2.

FIG. 14 is a representative chromatogram showing separation of R-NACA and S-NACA.

FIG. 15 shows XRPD of NPI-001 Form 1, NPI-001/urea pattern 1 and NPI-001/urea pattern 1 Crash Cool.

FIG. 16 shows XRPD Diffractograms of NPI-001 Form 1, diNACA, Sodium Methoxide, NPI-001/sodium methoxide pattern 1 from methanol and NPI-001/sodium methoxide pattern 1 from ethanol.

### DETAILED DESCRIPTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims. The present disclosure is directed to novel methods for making N-Acetyl Cysteine Amide (NACA), and novel intermediates thereof. More particularly, the present disclosure takes advantage of the novel intermediates to significantly, and surprisingly, increase the efficiency of preparing NACA in both a high chemical yield and a high enantiomeric purity. Below is the basic chemical structure of NACA.

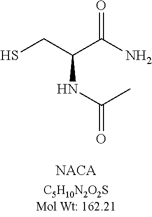

The following procedures may be employed for the preparation of the compound of the present disclosure. The starting materials and reagents used in preparing these compounds are either available from commercial suppliers such as the Aldrich Chemical Company (Milwaukee, Wis.), Bachem (Torrance, Calif.), Sigma (St. Louis, Mo.), or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supplements, Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989, relevant portions of which are incorporated herein by reference.

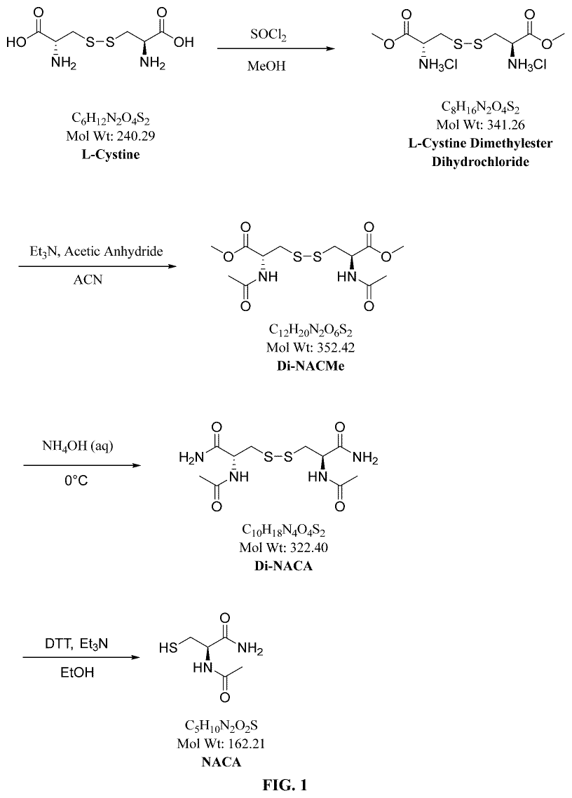

NACA is manufactured in 4 steps as shown in FIG. 1. The starting material is the naturally occurring L-cystine with the L conformation on both cysteine subunits. In the first step the two acid groups are protected by forming the di-methyl ester of L-cystine as the dihydrochloride salt. Alternatively, the synthesis may begin with commercially acquired L-cystine dimethyl ester dihydrochloride. In the second step the two nitrogens on the L-cystine are reacted with acetic anhydride to give diNACME which is in essence L-cystine with both acids protected as methyl esters and both primary amine groups protected with acetyl groups. Step 3 converts the methyl ester groups to primary amides. Step 4 reductively cleaves the diNACA intermediate to give NACA.

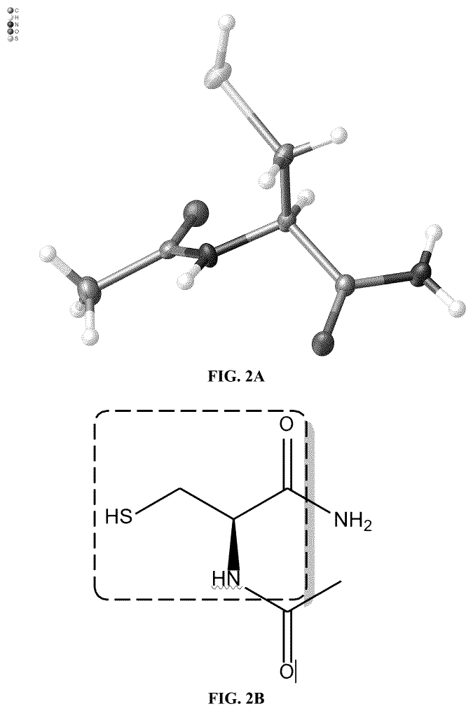

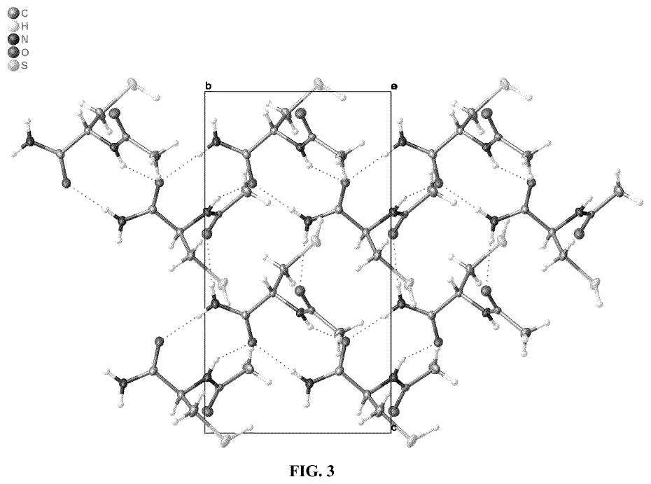

The preparation of NACA has one starting material that contributes significantly to the backbone of the final structure: L-cystine. In FIG. 2B, the portion of NACA that is in the dotted box is derived from L-cystine. L-Cystine provides the chiral center and the amino acid core. Two common reagents, acetic anhydride and ammonium hydroxide, provide atoms by derivatizing the carboxylic acid and primary amine, respectively.

TABLE 1
List of Starting Materials.

| Starting Material | Quality | Other |
|---|---|---|
| L-cystine | ≥98.5% pure | BSE/TSE-certification |

Control of Starting Materials. The quality of L-cystine may be controlled based on the specification provided by the manufacturer and/or incoming testing performed by the Contract Manufacturing Organization (CMO) as shown in Table 2.

TABLE 2
L-Cystine Testing.

| Attribute | Test Method | Specification/Limit |
|---|---|---|
| Appearance | Visual inspection | White powder |
| ID | NMR (USP<761>; D2O) | Conforms to structure |
| Purity¹ | Titration | 98.5-101.0% |
| Optical Rotation | Vendor method | −215 to −225° |

¹A use test may be substituted for the purity test

Reagents and Solvents. A list of reagents and solvents used in each step of the manufacturing process is provided in Table 3 and Table 4, respectively.

TABLE 3
List of Reagents

| Step | Reagent | Quality |
|---|---|---|
| 1 | Thionyl chloride | ≥97% |
| 2, 4 | Triethylamine | ≥99% |
| 2 | Acetic anhydride | ≥99% |
| 2 | Sodium bicarbonate | ≥99% |
| 2 | Sodium sulfate (anhydrous) | — |
| 3 | Ammonium Hydroxide | 28-32% |
| 4 | Dithiothreitol | ≥98% |

TABLE 4
List of Solvents.

| Step | Solvent | Quality |
|---|---|---|
| 1 | Methanol | ≥99% |
| 1,4 | methyl t-Butyl Ether (MTBE) | ≥99% |
| 2 | Acetonitrile | ≥99% |
| 2 | Ethyl acetate | ≥99% |
| 3,4 | EtOH | 89.0-92.0% |

In the first step, the dimethyl ester of L-cystine was formed. L-cystine and methanol were cooled to −10° C. before slowly adding thionyl chloride. The skilled artisan will recognize methanol can be substituted with other similar reactive alcohols, and/or other alternative carboxylic acid activating agents (e.g., oxalyl chloride, CDI, etc). The slurry was then heated to reflux to produce a solution. Initially, the reaction was held for 4 hours before proceeding to the workup. Using HPLC it was found to not achieve full conversion. Holding the reaction at reflux for 16 hours proved to be effective for conversion. Upon verifying reaction completion, the solution was solvent-exchanged into ethyl ether. Depending on the method of making, other such solvent may also be used, e.g., methyl tert-butyl ether (MTBE). MTBE has proven to be an effective replacement for ethyl ether, as such the solution can be solvent-exchanged successfully into MTBE, and can also be used for washes. The white solids were filtered and washed with MTBE before drying at 45° C. under vacuum to a yield of 95-99% with a purity of 96-99%.

In Step 2, the L-cystine dimethylester dichydrochloride is converted to di-N-acetylcystine dimethylester, DiNACME. L-cystine dimethylester dihydrochloride, as a slurry in acetonitrile, is cooled to 0±5° C. To the cooled solution is first added at least 4 equivalents of triethylamine followed by at least 2 equivalents of acetic anhydride while maintaining an internal temperature of ≤5° C. throughout the additions. After aging for not less than 30 minutes, reaction completion is confirmed by HPLC. To the reaction mixture is added ethyl acetate and an aqueous workup is performed. Upon completion of the aqueous workup, the organic solution is fully exchanged into ethyl acetate by vacuum distillation of the acetonitrile and replacement with ethyl acetate, several times. The product, di-N-acetylcystine dimethylester, is isolated by filtration and dried to a 73-75% yield with a purity of 93-97%. Step 2 can be accomplished in a couple of different ways. While not as efficient, the L-cystine dimethylester dihydrochloride was free-based using 300 weigh percent amberlyst-A21 in 10 volumes acetonitrile. The solution of free-based material in acetonitrile was then acetylated using 2.1 equivalents acetic anhydride and 2.5 equivalents triethylamine at room temperature for 1 hour. The crude material was concentrated and dissolved into ethyl acetate before being washed with saturated sodium bicarbonate and brine solutions. The washed organics were concentrated to dryness for a 73-75% yield with a purity of 93-97%.

Alternatively, and more efficiently, the Step 2, triethylamine after the free-basing was replaced with amberlyst-A21 for a total of 600 weight percent. The acetonitrile was increased to 15 volumes to facilitate agitation. After free-basing is complete, the acetic anhydride can be added at room temperature or below. Some impurities may be formed when reaction was performed at room temperature, as such, the reaction can be cooled to 0° C. to successfully reduce impurity formation. After reaction completion, the solution was previously concentrated to dryness. Typically, filterable solids may be preferred in certain isolation steps. As such, the acetonitrile solution was solvent-exchanged into ethyl acetate to yield filterable solids. These solids were filtered and washed with ethyl acetate before drying at 45° C. under vacuum for a 45% yield with a purity of 98%. The solvent-exchanged material can also be chilled to 0° C. for 1 hour to increase yield to 65% while achieving similar purity.

Typically, triethylamine is cheaper than amberlyst-A21, as such the reaction can also be accomplished using only triethylamine. This was achieved by cooling the mixture of L-cystine dimethylester dihydrochloride in 15 volumes acetonitrile to 0° C. before adding 4.2 equivalents of triethylamine followed by 2.1 equivalents of acetic anhydride. Previously, using amberlyst-A21 to free-base, the hydrochloride salt formed was bound to amberlyst-A21 and filtered off of the material to yield a clear colorless L-cystine dimethylester solution. Using triethylamine as free base led to the formation of triethylamine chloride salts. These were filtered off before salting out triethylamine using 0.5 N HCl in 13% NaCl solution. At this point, the reaction was solvent-exchanged into ethyl acetate and filtered similar to previous methods. The alternative method resulted in 88-92% yield with 93-95% purity.

Using only triethylamine for step 2 led to some loss of product before acid treatment, ineffective removal of triethylamine using acid, and degradation of material. When the triethylamine acetylation was first performed, solids were filtered before treating the solution. However, HPLC of the solids revealed the presence of desired product, Di-NACMe. Upon reaction completion, the slurry was now dissolved in 15 volumes ethyl acetate before treatment to maximize Di-NACMe in solution. Initially, 0.5 N HCl in 13% brine solution was used to treat the reaction solution. ¹H-NMR results of acid-treated product revealed significant amounts of triethylamine were still present in step 2 product. In addition to the ineffective removal of triethylamine, the presence of acid in the material before drying degraded the material into a brown taffy when heat was applied. Since acid not only failed to remove triethylamine but also degraded material, acid was avoided. Saturated sodium bicarbonate replaced HCl to free-base triethylamine, acetic acid and HCl after reaction completion. After treating reaction solution with base to neutralize HCl, the belief was that triethylamine could be azeotroped with acetonitrile (ACN) while acetic acid stayed behind in ethyl acetate after the solvent-exchange.

The amidation of Di-NACMe into diNACA was performed in ammonium hydroxide. Initially, the solid was charged with at least 3 equivalents of 28-30% aqueous ammonium hydroxide. The solids dissolved in solution as diNACA precipitated. The slurry was agitated for 2 hours after solid formation before filtering and washing with minimal water. The solids were dried at 45° C. under vacuum. This method resulted in 60-70% yield with 70% purity. A solvent-exchange into ethanol after reaction completion further increased yield without sacrificing purity. higher purity of the diNACA can be used to achieve a higher yield in the final reduction step. Additionally, diNACA may be recrystallized from water to further enhance the purity.

The reduction of diNACA to NACA can be accomplished using tris (2-carboxyethyl)phosphine (TCEP), thioglycolic acid, and/or dithiothreitol (DTT). One reduction method uses 1.1 eq TCEP in 15 volumes 10:1 THF:water, heated to reflux. Reduction by thioglycolic acid was performed using 2.5 eq thioglycolic acid. The reaction was attempted using THF and ethanol as solvents. THE reaction yielded ˜42% with a purity of 4%. Ethanol reaction had a yield of 2% with purity of 30%. With the goal of using ethanol, amberlyst-A21 and triethylamine were evaluated as bases to aid reduction. Amberlyst-A21 was determined to be a better base with yields of 45% and purity of 85%. Amberlyst-A21 also reduced reaction time from 2 days in triethylamine-mediated reactions to 3 hours in amberlyst-A21-mediated reactions. Alternatively, DTT can also be used. Various possible solvents can be used, e.g., dichloromethane (DCM), methanol, isopropanol, ethanol and/or other alcoholic solvents. Ethanol was chosen for its greater solubility of diNACA. The use of alternative reducing agents such as, but not limited to triphenylphosphine, TCEP, thioglycolic acid, etc., is contemplated herein as well as solvents such as, but not limited to, THF, EtOH, iPrOH, MeOH, DCM, water, etc. and bases, whether catalytic or stoichiometric, such as, but not limited to, Et3N, NaOH, amberlyst-A21, etc. Initially, to effect the reduction of the disulfide bond, 1.5 equivalents triethylamine were added to diNACA dissolved in ethanol followed by 1.5 equivalents of DTT. The solution was heated to reflux and held for 2 hours before solvent-exchanging into methyl tert-butyl ether to precipitate filterable solids. Further optimization of the reaction led to the reduction of triethylamine to a catalytic 0.1 equivalents, DTT to 1.25 equivalents and the reaction temperature to 62±3° C. with the effect of improving the impurity profile of the isolated NACA. This method yielded 85% of NACA with a purity of ≥98.0%. The purity of NACA may be further enhanced via recrystallization from ethanol.

These efforts to increase purity focused on the major impurities as measured by HPLC, namely, diNACA, DTT and cyclic DTT. Oxidation of NACA occurs when exposed to air. Ethanol and methyl t-butyl Ether (MTBE) used in the reduction and solvent-exchange were degassed in an effort to reduce diNACA. MTBE proved to be efficient in removing DTT and cyclic DTT, so an MTBE trituration was performed to improve purity.

diNACA purifications were performed with the goal of taking purer material through reduction for purer NACA.

Step 1: Formation of L-Cystine Dimethylester Dihydrochloride.

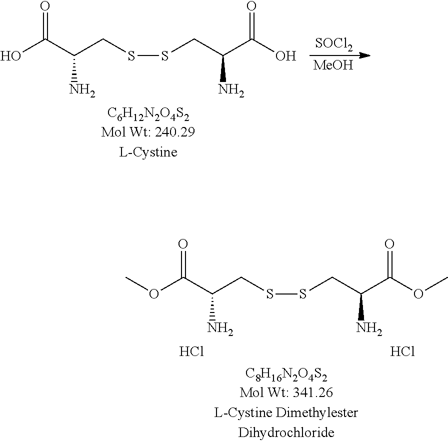

| Reagents/ | | | | | Weight/ | |
|---|---|---|---|---|---|---|
| Materials | MW | Density | Eqs. | Moles | Volume | |
| L-Cystine, | 240.29 | — | 1.0 | 208 | 50 | kg |
| ≥98.5% | | | | | | |
| Thionyl | 118.97 | 1.64 | 2.41 | 504 | 60 | kg |
| Chloride, ≥97% | | | | | | |
| Methanol | 32.04 | 0.79 | 12.5 vol | — | 625 | L |
| (MeOH), ≥99% | | | | | | |
| Methyl-tert | 88.15 | 0.74 | 8 vol | — | 400 | L |
| Butyl Ether | | | | | | |

(MTBE),
≥99%

Set-up: A 2000 L glass-lined reactor;

50 kg L-Cystine; and

625 L Methanol was charged to the reactor and agitated while cooling to −10° C.

60 kg Thionyl Chloride was slowly added at ≤−5° C. After addition completion, the reaction material was heated to reflux and held for 16 hours. After the reaction was verified as complete by HPLC (≤0.5% starting material), the reaction was cooled to room temperature. The reaction mixture was concentrated to 6 volumes before solvent-exchanging into 3×8 volumes MTBE. The resulting slurry was agitated at room temperature for 1 hour before being filtered and washed with MTBE. The solids were dried at 45° C. under vacuum.

Yield: 68.15 kg (95.9%), Purity: 95.8%.

Step 2: Formation of di-N-acetyl-l-cystine dimethylester (Di-NACMe).

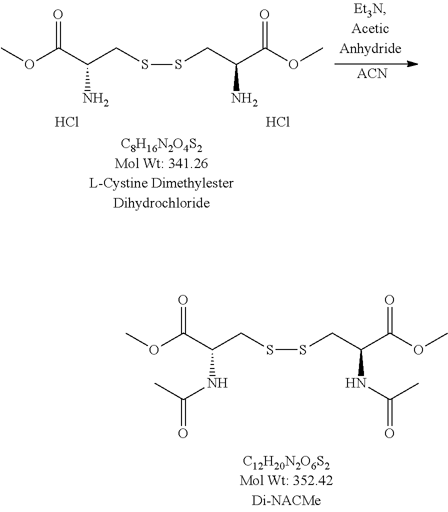

| | | | | | Weight/ |
|---|---|---|---|---|---|
| Reagents/Materials | MW | Density | Eqs. | Moles | Volume |
| L-Cystine Dimethylester | 341.26 | — | 1.0 | 76.2 | 26 |
| Dihydrochloride, ≥95% | | | | | |
| Acetonitrile, ≥99% | 41.05 | 0.79 | 23 vol | — | 472 |
| Triethylamine (TEA), ≥99% | 101.19 | 0.73 | 4.2 | 316.2 | 32 |
| Acetic Anhydride, ≥99% | 102.09 | | 2.1 | 156.7 | 16 |
| Ethyl Acetate, ≥99% | 88.1 | | 41 vol | — | 958 |

Set-up: A 800 L glass lined reactor.

26 kg L-Cystine Dimethylester Dihydrochloride and

390 L Acetonitrile (ACN) is charged to the reactor and agitated while cooling to 0° C.

32 kg Triethylamine is added to the reactor at ≤5° C.

16 kg Acetic Anhydride is slowly added to the reactor at ≤5° C. Upon addition completion the reaction is held for 30 minutes at 5±5° C. After reaction was verified as complete by HPLC (≤0.5% starting material), 10 volumes ethyl acetate was charged to the reactor and agitated to ambient. The resulting reaction mixture was washed with 2×2 volumes saturated bicarbonate. The aqueous layer was back extracted with 5 volumes ethyl acetate. All organics were combined and dried over sodium sulfate and polish filtered. The filtrate was concentrated to 5 volumes before azeodrying with 2×4 volumes acetonitrile followed by a solvent-exchange into 4×6 volumes ethyl acetate. The resulting slurry was agitated at 0° C. for 1 hour before being filtered and washed with 2 volumes ethyl acetate. The solids were dried at 25° C. under vacuum.

Average Yield: 29.56 kg (100%), Average Purity: 92.2%.

Step 3: Formation of DiNACA.

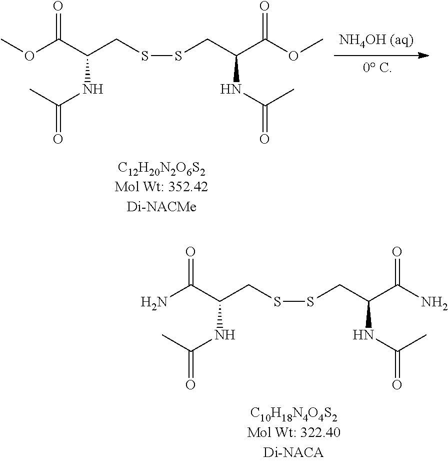

| | | | | | Weight/ | |
|---|---|---|---|---|---|---|
| Reagents/Materials | MW | Density | Eqs. | Moles | Volume | |
| Di-NACMe | 352.42 | — | 1.0 | 247 | 87.05 | kg |
| 28-30% Ammonium | 35.05 | 0.9 | 8.44 | 2088 | 244 | kg |
| Hydroxide | | | | | | |
| Ethanol, absolute | 46.07 | 0.79 | 17 vol | — | 1483 | L |
| 200 proof | | | | | | |

Set-up: A 800 L glass lined reactor.

244 kg Degassed 28-30% NH4OH (aq) was cooled to 0° C. before.

87.05 kg Di-NACMe was charged to the reactor and agitated for 4 hours. After reaction was verified as complete by HPLC (≤0.5% starting material), the reaction mixture was solvent-exchanged into 3×5 volumes degassed ethanol. The resulting slurry was agitated at 0° C. for 30 minutes before being filtered and washed with cold degassed ethanol. The solids were dried at 45° C. under vacuum.

Average Yield: 52 kg (67.1%), Average Purity: 72.0%.

Step 3A: Purification of DiNACA.

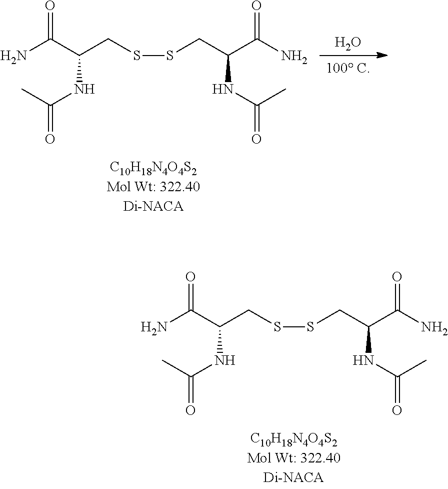 [SMILES: CC(=O)N[C@@H](CSSC[C@H](NC(C)=O)C(N)=O)C(N)=O ; O ; CC(=O)N[C@@H](CSSC[C@H](NC(C)=O)C(N)=O)C(N)=O]

| | | | | | Weight/ | |
|---|---|---|---|---|---|---|
| Reagents/Materials | MW | Density | Eqs. | Moles | Volume | |
| DiNACA | 322.40 | — | 1.0 | 161 | 52 | kg |
| Process Water, Filtered | 18.02 | 1.0 | 8 vol | — | 416 | L |

Set-up: A 800 L glass lined reactor.

52 kg DiNACA and

416 L Degassed Water were charged to the flask and agitated while heating to reflux. Upon dissolution, the reaction solution was allowed to cool overnight. The solids were filtered and washed with 2 volumes cold degassed water. The solids were dried at 45° C. under vacuum.

Recovery: 36 g (69%), Purity: 86.4%.

Step 4: Formation of NACA.

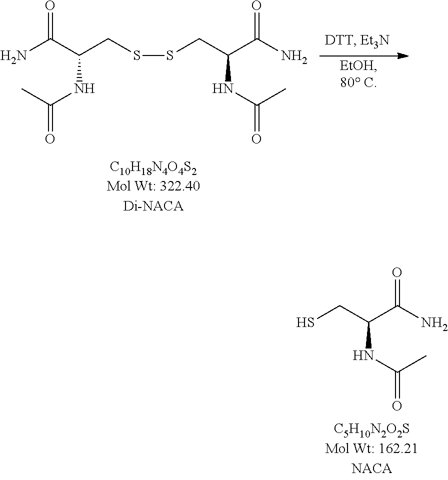

| | | | | | Weight/ | |
|---|---|---|---|---|---|---|
| Reagents/Materials | MW | Density | Eqs. | Moles | Volume | |
| DiNACA | 322.40 | — | 1.0 | 112 | 36 | kg |
| Ethanol, absolute, | 46.07 | 0.79 | 20 vol | — | 720 | L |
| 200 proof | | | | | | |
| Triethylamine | 101.19 | 0.73 | 0.1 | 8.8 | 1.1 | kg |
| (TEA), ≥99% | | | | | | |
| Dithiothreitol, ≥98% | 154.25 | — | 1.25 | 143 | 22 | kg |
| (aka 1,4-Dithiothreitol | | | | | | |
| and DL-Dithiothreitol) | | | | | | |

Set-up: A 800 L glass lined reactor.

720 L Degassed Ethanol,

1.1 kg Triethylamine,

22 kg Dithiothreitol and

36 kg of DiNACA were charged to the reactor before heating the reaction to 62±3° C. The reaction is held at temp for 2 hours. After reaction was verified as complete by HPLC (≤0.5% starting material on overloaded column), cool reaction to ambient. The reaction solution was polish filtered and concentrated to 10 volumes before solvent exchanging into 4×10 volumes degassed MTBE. The resulting slurry was agitated overnight before being filtered and washed with 2 volumes degassed MTBE. The solids were dried @45° C. under vacuum. diNACA was recrystallized with ethanol.

Yield: 27.8 kg (77.2%), Purity: 98.5%.

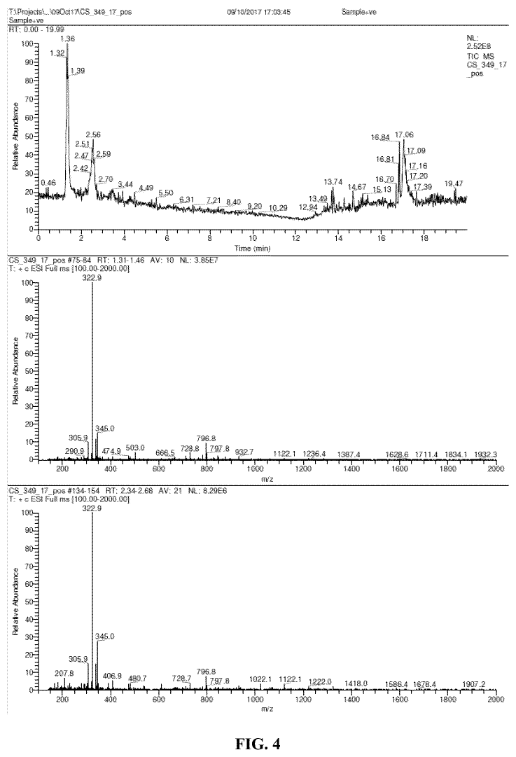

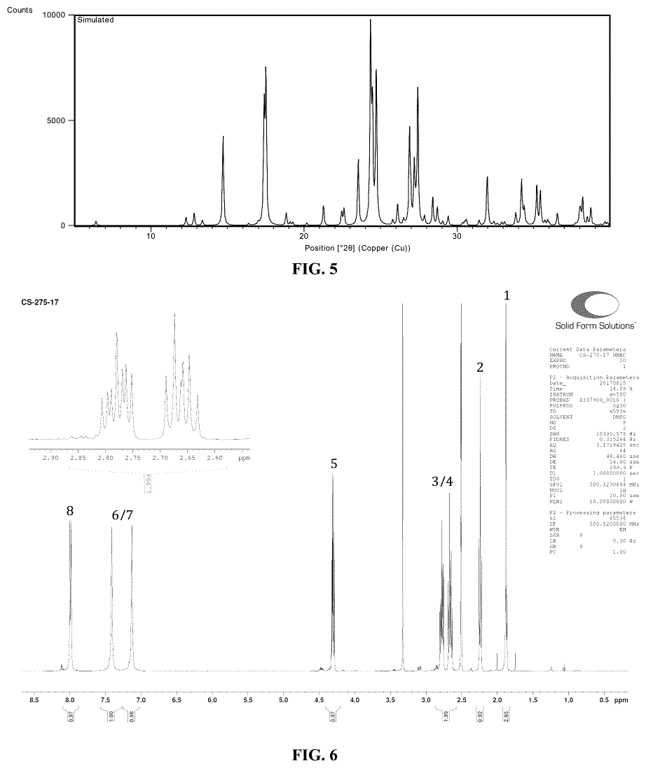

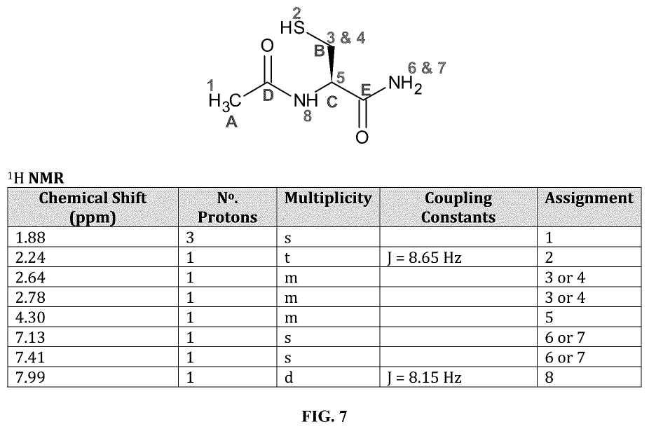

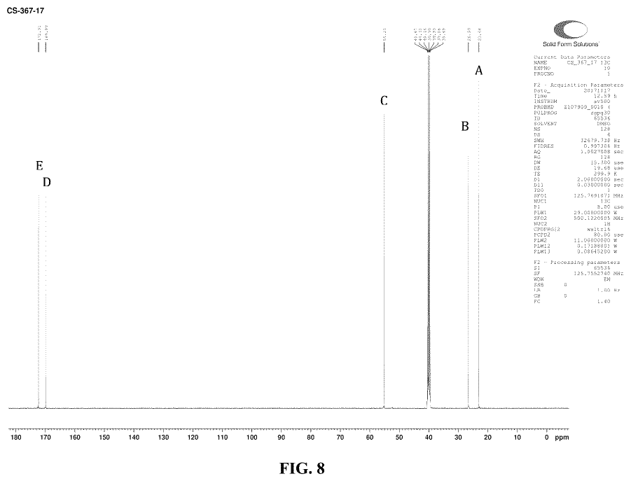

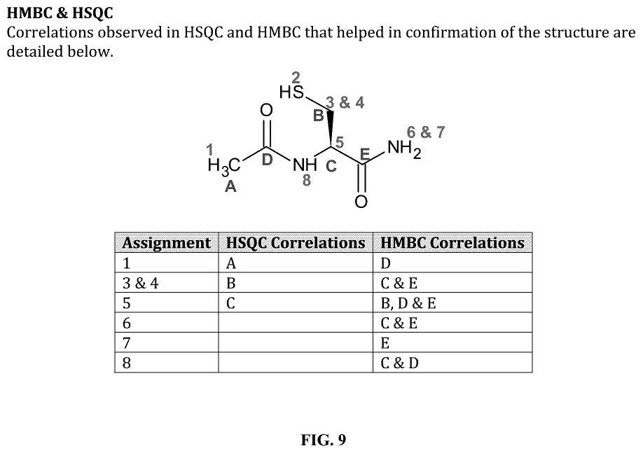

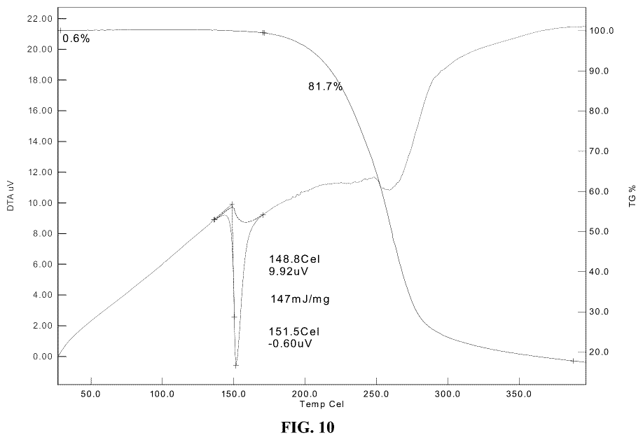

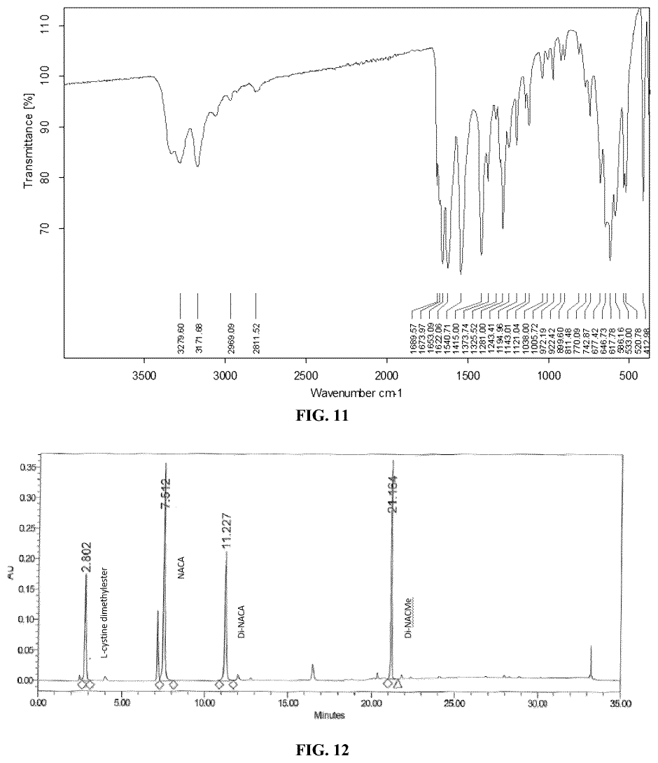

FIG. 1 shows a basic chemical synthesis of the present disclosure. FIG. 2A shows a view of NPI-001 molecule ‘A’ without atom labeling. All non-hydrogen atoms are shown with thermal ellipsoids set at the 50% probability level. Color code: Carbon, grey; H, white; O, red; S, yellow. FIG. 2B shows a portion of NACA derived from starting material L-Cystine. FIG. 3 shows a view of unit cell an axis of NPI-001 containing complete molecules. All atoms are shown with thermal ellipsoids set at the 50% probability level. Color code: Carbon, grey; H, white; O, red; S, yellow. FIG. 4 shows results of liquid chromatography with mass spectrometric detection. FIG. 5 shows Simulated (120 K) XRPD 2θ diffractogram of NACA. FIG. 6 shows ¹H-NMR of NACA. FIG. 7 shows ¹H-NMR assignments for NACA (¹H and ¹³C assignments are based on analysis of the 1D ¹H NMR, ¹H-¹³C HSQC and ¹H-¹³C HMBC spectra.). FIG. 8 shows ¹³C-NMR of NACA. FIG. 9 shows ¹³C-NMR assignments for NACA. (¹H and ¹³C assignments are based on analysis of the 1D ¹H NMR, ¹H-¹³C HSQC and ¹H-¹³C HMBC spectra). FIG. 10 shows combined thermogravimetricy/differential scanning calorimetry scans of NACA. FIG. 11 shows fourier transform-infrared spectrum (FT-IR) of NACA.

Analytical procedures for NACA. The analytical methods for the testing of NACA drug substance are listed in Table 5. Most of the methods are USP compendial tests. The additional NACA drug substance methods (which are not compendial) include two HPLC methods for assay and impurities, and one chiral HPLC method for chiral purity. Each of the non-compendial methods is described in more detail in sections that follow.

TABLE 5
List of Analytical Procedures for NACA Drug Substance

| Test | Test Method | Description |
|---|---|---|
| Appearance | Visual | A sample of the solid material |
| | | is examined visually for form |
| | | and color. |
| ID-1 | IR | Method follows USP<197A> |
| ID-2 | ¹H-NMR | Method follows USP<761> |
| Potency/Assigned | Calculated | Purity = (100 − % HPLC |
| Purity | | impurities − % H₂O − % |
| | | ROI − % Total Residual |
| | | Solvents) |
| Organic | HPLC-Method I | Reverse phase gradient HPLC |
| Impurities/Related | | method. |
| Substances | HPLC Method II | Reverse phase gradient HPLC |
| | | method. |
| Chirality | Optical Rotation | Method follows |
| | | USP<781> (c 1.00, MeOH) |
| Chiral Purity | Chiral HPLC | Chiral HPLC method |
| Residue on | USP<281> | Method follows USP<281> |
| Ignition | | |
| DSC | USP<891> | Method follows USP<891> |
| X-ray Powder | USP<941> | Method follows USP<941> |
| Diffraction | | |
| Heavy Metals | USP<233> | Method follows USP<233> |
| Residual Solvents | GC (USP<467>) | Method follows USP<467> |
| Water Content | Karl Fischer | Method follows USP<921> |
| | | version 1c |
| Microbial Limits | Microbial | Method follows USP<61> |
| | enumeration | |
| | Test for specified | Method follows USP<62> |
| | organisms | |

HPLC Method for Purity and Related Substances. Analysis of the NACA drug substance for purity and related substances makes use of a reverse phase HPLC with an ultraviolet (UV) detector. The method is summarized in Table 6. This method is also used as the in-process method for each step to follow completion of reaction.

TABLE 6
Summary of NACA HPLC Method I
(Purity and Related Substances)

| Method Element | Description | | |
|---|---|---|---|
| Column | Phenomenex Synergi Hydro-RP, | | |
| | 4.6 × 250 mm, 4 μm | | |
| Detection | 214 nm | | |
| Column Temperature | 40° C. | | |
| Injection Volume | 25 μL | | |
| Flow Rate | 1.0 mL/minute | | |
| Mobile Phase A | 0.02% H₃PO₄ in H₂O | | |
| Mobile Phase B | Acetonitrile (ACN) | | |
| Gradient | Time (min) | % A | % B |
| | 0.0 | 100 | 0 |
| | 15.0 | 90 | 10 |
| | 25.0 | 0 | 100 |
| | 30.0 | 0 | 100 |
| | 30.1a | 100 | 0 |
| | 35.0 a | 100 | 0 |

| | aEquilibration time—no integration |
|---|---|
| System Suitability | 1. Specificity: No significant interference in |
| | the blank chromatogram at retention times |
| | of interest. |
| | 2. The S/N of the NACA peak in the 0.03% |
| | sensitivity |
| | solution must be ≥ 10 |
| | 3. The % RSD of the RT and peak area of NACA |
| | in the 5 |
| | injections of the first sample must be ≤ 2.0%. |
| | 4. The recovery of one standard prep (average |
| | of all injections) versus a second standard |
| | prep (average of all injection) must be 100.0 ± |
| | 2.0%. |

FIG. 12 shows a Method I Chromatogram Showing NACA and all Intermediates and Starting Material.

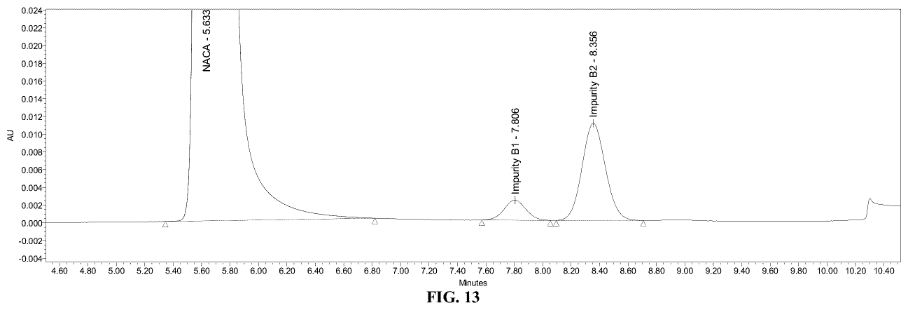

HPLC Method for Impurities B1 and B2. Method-I did not always detect impurities B1 and B2. A second method, Method-II, was developed to reliably quantitate these two impurities. The method is summarized in Table 7. A representative chromatogram showing the retention times of B1 and B2 is presented in FIG. 13.

TABLE 7
Summary of NACA HPLC Method II
(Impurities B1 and B2)

| Method Element | Description | | |
|---|---|---|---|
| Column | Agilent Zorbax SB-Aq, | | |
| | 4.6 × 250 mm, 5 μm | | |
| Detection | 214 nm | | |
| Column Temperature | 40° C. | | |
| Injection Volume | 25 μL | | |
| Flow Rate | 1.0 mL/minute | | |
| Mobile Phase A | 0.02% H₃PO₄ in H₂O | | |
| Mobile Phase B | Acetonitrile (ACN) | | |
| Gradient | Time (min) | % A | % B |
| | 0.0 | 100 | 0 |
| | 5.0 | 100 | 0 |
| | 20.0 | 0 | 100 |
| | 25.0 | 0 | 100 |
| | 25.01 | 100 | 0 |
| | 35.0 | 100 | 0 |

| | |
|---|---|
| System Suitability | 1. Specificity: No significant interferences in |
| | the blank chromatogram at retention times |
| | of interest. |
| | 2. The S/N of the NACA peak in the 0.03% |
| | sensitivity |
| | solution must be ≥ 10 |
| | 3. The % RSD of the RT and peak area of NACA |
| | in the 5 |
| | injections of the first sample must be ≤ 2.0%. |

FIG. 13 is a representative Chromatogram of Method-II Showing B1 and B2

Chiral HPLC Method for Measuring Chiral Purity of NACA. A chiral method was developed to assess the chiral purity of NACA. The method is summarized in Table 8.

TABLE 8
Summary of Chiral HPLC Method for NACA

| Method Element | Description |
|---|---|
| Column | Diacel Chiralpak IC-3, |
| | 4.6 × 150 mm |
| Detection | 217 nm |
| Column Temperature | 35° C. |
| Injection Volume | 20 μL |
| Flow Rate | 0.8 mL/minute |
| Mobile Phase | 0.05% H₃PO₄ in 1:1 n-hexane:IPA |
| (isocratic) | |
| System Suitability | 1. Specificity: No significant interferences in the |
| | blank chromatogram at retention times of |
| | interest. |
| | 2. The resolution between the enantiomers is |
| | sufficient to allow for accurate integration of |
| | both peaks. |
| | 3. The S/N of the NACA peak in the 0.03% |
| | sensitivity solution must be ≥ 10 |
| | 4. The % RSD of the RT and peak area of NACA |
| | in the 6 injections of the first sample must |
| | be ≤ 2.0%. |

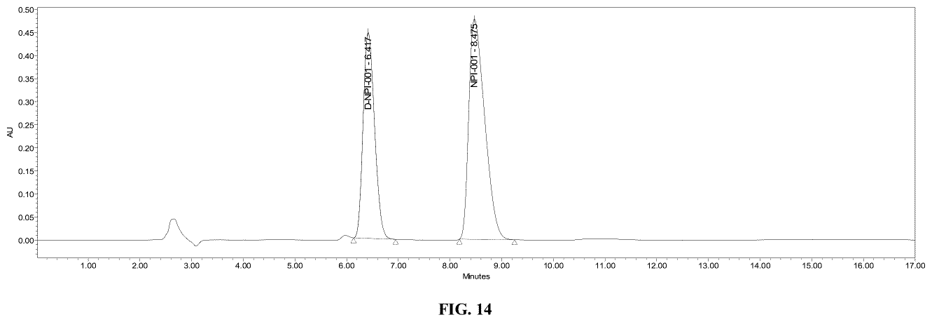

FIG. 14 is a chromatogram Showing Separation of R-NACA and S-NACA.

Preparation of D-NACA. D-Cystine was obtained from a commercial vendor. D-Cystine was dissolved in water and pH was adjusted to pH 9-10 with NaOH. Acetic anhydride was added dropwise at 0° C. and pH maintained at 9-10. Solution was stirred for 4 hours after which it was acidified to pH ˜2 with HCl. The solvent was evaporated under reduced pressure. 20 mL MeOH was added to the flask to dissolve contents. Solution was filtered. Filtrate was concentrated and evaporated. Thin layer chromatography (MeOH:DCM:HOAc, 1:8:1) indicated loss of starting material and appearance of a single new peak for D-acetylcystine. D-Acetylcystine was charged to a round-bottomed flask with 50 mL MeOH to which was added 0.35 mL concentrated H₂SO₄ via syringe, dropwise, at ambient temperature, with agitation. Solution turned turbid. IPC by TLC (MeOH:DCM, 1:9] showed no starting material. Solvent was evaporated. Fraction was neutralized with NaHCO3 (saturated), extracted with DCM (50 ml twice), washed with water, dried over Na2SO4, filtered, purged with nitrogen and concentrated under reduced pressure to yield a net weight of 7.8 g white solid, formed in the refrigerator. N-acetylcysteine methyl ester was charged into a 3-necked, round-bottomed flask with nitrogen bubbler, and magnetic stirrer. Ammonium hydroxide at ambient temperature was added and purged with nitrogen through reaction mixture and agitated at ambient temperature. Solvent was evaporated under vacuum and a white solid formed. Ethanol was added and heated to form clear solution and left to stand overnight. White solid crystallized, was filtered, washed with ethanol and purified by column chromatography.

Oral Solution of NACA.

A study was conducted to determine the solubilization of NACA in ORA-SWEET® (Ora-Sweet is a commercially available syrup vehicle containing water, sucrose, glycerin, sorbitol, flavoring, buffering agents (citric acid and/or sodium phosphate), methyl paraben and potassium sorbate, pH 4.2 manufactured by Paddock Laboratories, Inc., Minneapolis, Minnesota). HPLC equipment and glass containers and stirrers were utilized.

NACA at 80 mg/ml did not readily dissolve in ORA-SWEET, rather it required stirring for 20-30 minutes to achieve a solution. However, NACA was readily dissolved in water with shaking for 30 seconds, followed by dilution with an equal volume of ORA-SWEET with shaking for 20-30 seconds. Therefore, NACA was dissolved in 50 mL water followed by 50 mL Ora-Sweet, shaken to dissolve in an opaque plastic bottle with closure and provided to subject for self-administration (oral ingestion).

NACA oral solution was also be prepared as 100 ml oral solution in ORA-SWEET. Doses can be achieved ranged from 250 mg to 4000 mg/day/patient. The NACA dissolution in water followed by dilution with ORA-SWEET was found optimal for compounding. ORA-SWEET is a pale pink solution with a cherry syrup flavor. NACA has a mild sulfur odor and a bitter taste (like burned sesame seeds). When dissolved in ORA-SWEET, the odor and taste were masked.

The following instructions for preparation of NACA Oral Solution were developed:

Weigh NACA [either 250, 750, 1500, 3000 or 4000 mg (±1 mg), as appropriate for the particular dose group] and place into a 125 mL (approximately) capacity opaque high density polyethylene, labeled (see Table 9) bottle with opaque polypropylene screw cap.

TABLE 9
Container/closure for NACA
Oral Solution Used for Phase 1 Study

| Component | Description |
|---|---|
| Bottle | 125 mL opaque white high |
| | density polyethylene bottle |
| Cap | Polypropylene opaque white cap |
| Label | Pharmacy approved label |

Measure 50 mL of Purified Water and pour into each bottle containing NACA and shake vigorously by hand (at least 30 seconds) to dissolve.

Measure 50 mL Ora-Sweet and pour into each bottle containing NACA and shake vigorously by hand (at least 30 seconds) to dissolve.

A subject drinks entire solution, followed by two 20-ml rinses of the container with water, which are also drank.

Tablet or Capsule of NACA for clinical phase 1 or clinical phase II trials:

The qualitative composition for NACA Tablets is presented in Table 10.

TABLE 10
Qualitative Composition of NACA Tablets

| Component | Quality Standard |
|---|---|
| NACA | Nacuity |
| Lactose | NF |
| Microcrystalline Cellulose | NF |
| Croscarmellose Sodium | NF |
| Stearic acid | NF |

NACA Tablets, 250 mg, are formulated as an immediate release drug product. A roller-compacted blend containing 250 mg NACA is compressed into round, biconvex tablets.

The formulation used to manufacture NACA Tablets is a roller compacted blend of common excipients (Table 11). The blend is compressed into round, biconvex shaped tablets.

TABLE 11
Quantitative Composition of NACA Tablets

| | mg/tablet | | | Quality |
|---|---|---|---|---|
| Component | weight | % | Function | Standard |
| NACA | 250.0 | 50 | Drug | Nacuity |
| | | | substance | |
| Lactose | 42.875 | 8.575 | Filler | NF |
| Microcrystalline | 177.125 | 35.425 | Filler | NF |
| Cellulose | | | | |
| Croscarmellose | 25 | 5.0 | Disintegrant | NF |
| Sodium | | | | |
| Stearic acid | 5 | 1.0 | Lubricant | NF |
| TABLET WT | 500 | 100 | — | — |

Ranges for Components of NACA Tablets (requiring corresponding quantitative adjustments to other components).

NACA range: 1-55%.

Lactose range: 0-65%.

Microcrystalline cellulose range: 0-91%.

Croscarmellose Sodium range: 0-9%.

Steric Acid range: 0.1-5.6%.

Type of Container and Closure for Dosage Form.

NACA Tablets, 250 mg, are packaged in high density polyethylene (HDPE) bottles with foil induction seal and a white polypropylene screw-top closure. NACA-acceptable, biocompatible excipients used for the formulation meet compendial standards. Lactose functions as a filler. Microcrystalline cellulose functions as a filler. Croscarmellose Sodium functions as a disintegrant. Stearic Acid functions as a lubricant. Excipients were screened by assessing the stability of NACA in mixtures with each excipient. Mixtures of NACA with either microcrystalline cellulose, lactose monohydrate, croscarmellose sodium and crospovidone/Kollidon CL and hydroxypropyl cellulose exhibited less degradation of NACA compared to other excipients (Table 12). Hydroxypropyl cellulose exhibited greater levels of impurities than the other lead excipients (data not shown). Based on these results as well as the collective experience of the Formulators, NACA-acceptable, biocompatible excipients, microcrystalline cellulose, lactose monohydrate, croscarmellose sodium and stearic acid were chosen as excipients for NACA Tablets.

TABLE 12
Stability results for NACA/excipient mixtures after 4 weeks at
40° C./75% RH

| | | Ratio of | % |
|---|---|---|---|
| Sample ID | Excipient | API:Excipient | Assay |
| SPt: | NA. | 1:0 | 95.3 |
| 17004Q4; P | | | |
| SPL- | Microcrystalline Cellulose PH | 1:1 | 94.9 |
| 1700405-P | 102 | | |
| SPL- | Lactose Monohydrate | 1:1 | 93.7 |
| 1700406-P | (Fastflo) | | |
| SPL- | Croscarrnellose Sodium | 4:1 | 93.6 |
| 1700407-P | | | |
| SPL- | Crospovidone/Kollidon CL | 4:1 | 94.4 |
| 1700408-P | | | |
| SPI- | Sodium Lauryl Sulfate | 4:1 | 26.2 |
| 1700409-P | | | |
| SPL- | Colloidal Silicon Dioxide | 4:1 | 92.7 |
| 1700410-P | | | |
| SPL- | Magnesium Stearate | 9:1 | 77.5 |
| 1700411-P | | | |
| SPL- | Sodium Stearyl Fumarate | 9:1 | 86.7 |
| 1700412-P | | | |

Tables 13 and 14 were prepared. The use of roller compaction of formulations of NACA with various excipients yielded powders with acceptable flow properties. Roller compaction followed by compression yielded tablets with acceptable properties based on friability and hardness. The clinical formulation was selected based on acceptable 2-week stability data.

Dry blending of formulations of NACA with various excipients yielded poorly flowing powders. Dry blending followed by compression yielded tablets that were unsatisfactory based on friability and hardness.

The use of roller compaction of formulations of NACA with various excipients yielded powders with acceptable flow properties. Roller compaction followed by compression yielded tablets with acceptable properties based on friability and hardness.

TABLE 13
Finished Prototype NACA Tablets by Roller Compaction

| | | Packaging |
|---|---|---|
| Batch Formulation | Product Batch # | Configurations* |
| NAC1G50%0401 | NAC1T250mg0401A | 1 |
| Low Roller Compaction | NAC1T250mg0401B | 2 |
| Force (3 kN) | | |
| NAC1G50%0402 | NAC1T250mg0402A | 1 |
| High Roller Compaction | NAC1T250mg0402B | 2 |
| Force (6 kN) | | |
| NAC1G50%0501 | NAC1T250mg0501A | 1 |
| Low Roller Compaction | NAC1T250mg0501B | 2 |
| Force (3 kN) | | |
| NAC1G50%0502 | NAC1T250mg0502A | 1 |
| High Roller Compaction | NAC1T250mg0502B | 2 |
| Force (6 kN) | | |

*Packaging Configuration 1 (With Desiccant):
Bottle: 60 CC 33/400 W-HDPE ROUND BTL
Cap: 33 mm SECURX with SG-75M liner
Desiccant: Desiccant Canister Silica Gel 2GM
Fill: 20 tablets per bottle
*Packaging Configuration 2 (Without Desiccant):
Bottle: 60 CC 33/400 W-HDPE ROUND BTL
Cap: 33 mm SECURX with SG-75M liner
Fill: 20 tablets per bottle

TABLE 14
NACA Tablet Prototype Batch Formulations by Roller Compaction

| | NAC1G50% 0401 | | NAC1G50% 0402 | | NAC1G50% 0501 | | NAC1G50% 0502 | |
|---|---|---|---|---|---|---|---|---|
| | mg/tablet weight | | mg/tablet weight | | mg/tablet weight | | mg/tablet weight | |
| | Low Force | | High Force | | Low Force | | High Force | |
| Component | g | % | g | % | g | % | g | % |

INTRAGRANULAR

| | | | | | | | | |
|---|---|---|---|---|---|---|---|---|
| NACA | 750 | 50 | 750 | 50 | 750 | 50 | 0 | 0 |
| NACA Direct Blend 70% | 0 | 0 | 0 | 0 | 0 | 0 | 0 | 71.42 |
| (NAC1B70% 01) | | | | | | | | |
| Lactose | 330 | 22 | 330 | 22 | 128.6 | 8.57 | 857.0 | 0 |
| Microcrystalline Cellulose | 330 | 22 | 330 | 22 | 128.6 | 8.57 | 0 | 0 |
| Croscarmellose Sodium | 75 | 5 | 75 | 5 | 53.6 | 3.57 | 0 | 0 |
| Stearic Acid | 7.5 | 0.5 | 7.5 | 0.5 | 10.6 | 0.71 | 0 | 0 |

EXTRAGRANULAR

| | | | | | | | | |
|---|---|---|---|---|---|---|---|---|
| Microcrystalline Cellulose | 0 | 0 | 0 | 0 | 402.9 | 26.86 | 322.3 | 26.86 |
| Croscarmellose Sodium | 0 | 0 | 0 | 0 | 21.4 | 1.43 | 17.2 | 1.43 |
| Stearic Acid | 7.5 | 0.5 | 7.5 | 0.5 | 4.3 | 0.29 | 3.5 | 0.29 |
| Total | 1500 | 100 | 1500 | 100 | 1500 | 100 | 1200 | 100 |

*NAC1B70% 01 = 70% NACA + 12% Lactose + 12MCC + 5% Croscarmellose Sodium + 1% Stearic Acid

NACA Tablet Dry Blend Formulation Development.

NACA Tablet formulations (Table 15) were dry blended and directly compressed. The flow of formulation from the hopper to the press was not uniform. Also, the resulting tablets suffered poor friability, hardness and capping. As a result dry blending was abandoned.

TABLE 15
Composition of Prototype NACA
Tablet Dry Blend Formulations

| | Prototype Batch Composition | | | |
|---|---|---|---|---|
| | Batch | | Batch | |
| | NAC1B50%0101 | | NAC1B50%201 | |
| Component | g | % | g | % |
| NACA | 600 | 50 | 600 | 50 |
| Lactose | 528 | 44 | — | — |
| Microcrystalline Cellulose | — | — | 528 | 44 |
| Croscarmellose Sodium | 60 | 5 | 60 | 5 |
| Stearic Acid | 12 | 1 | 12 | 1 |
| Total | 1200 | 100 | 1200 | 100 |

The formulations described above can also be used as a dry blend for filling into capsules.

The relation between micronization conditions and an initial increase of the degradation product diNACA was further investigated. Comparing stainless steel and zirconium oxide grinding jars gives a clear indication that steel samples undergo a time dependent increase of the degradation product diNACA. In contrast, using zirconium oxide jars and balls did not lead to an initial increase of the degradation product diNACA. Therefore, the zirconium oxide milling process was further investigated and successfully optimized regarding particle size distribution, milling parameters and impurity profile for the 1% NACA formulation. Zirconium oxide milling process was investigated and successfully optimized regarding particle size distribution, milling parameters and impurity profile for the 1% NACA formulation. The particle size distribution by laser diffraction analysis was x₁₀=1.3 μm, x₅₀=4.7 μm and x₉₀=12.3 μm. Batches were prepared and found to be stable for up to 4 weeks at ambient temperature.

Single Crystal X-ray Diffraction.

The absolute structure of NACA has been determined by single crystal X-ray diffraction from suitable crystals grown from cooling of a saturated 2-propanol NACA solution to ambient conditions. Single crystal analysis of crystals clearly shows that the material is NACA with the expected bond connectivity. The absolute stereochemistry has been proved in the crystal with excellent confidence, as confirmed by the Flack parameter being −0.02(3). The density of the material is high, reducing the risk that a more stable polymorph is even possible and the hydrogen bonding network observed satisfies the expected functionality observed in NACA. The predicted XRPD from the SC-XRD data is consistent with the Form 1 material, indicating that the crystal was representative of the bulk material. Data was collected and found to be twinned, therefore, was refined accordingly using HKLF5 and BASF commands, locating a two component twin with BASF scales 0.7271(10):0.2729(10) in the Monoclinic space group P21 where two complete formula units of NACA were found in the asymmetric unit only. No disorder was noted in the final structure with final a R1 [I>2σ(I)] of 3.30% obtained with Flack parameter of −0.02 with e.s.d 0.03 determined using 1549 quotients that is suitable to accurately determine the IUPAC name of NACA as 2R)-2-(acetylamino)-3-sulfanylpropanamide (=N-acetyl-L-cysteine amide=(R)-2-acetylamino)-3-mercapto-propamide).

NACA, Form 1 overall structure quality factor: 1.

Where:
1. Strong data set, no disorder, R1˜4%. Publishable quality.
2. Good data set, contains some minor disorder, R1˜6%. Publishable quality.
3. Average data set and/or easily modelled disorder or twinning. Publishable with care.
4. Weak data and/or major disorder or twinning that is not easily modelled. Publishable in some cases.
5. Very weak data and/or unexplained features of data or model. Not of publishable quality.

Polymorphism.

A detailed polymorph screen of NACA (NACA) (NACA) has been performed using a variety of solvents and experimental conditions. During the primary screen, the most common solid form observed was pattern 1 (Table 16).

TABLE 16
Crystallographic parameters and refinement
indicators of NACA, Form 1.
NACA, Form 1

| Empirical formula | C₅H₁₀N₂O₂S |
|---|---|
| Formula weight | 162.21 |
| Temperature/K | 120(1) |
| Crystal system | Monoclinic |
| Space group | P2₁ |
| a/Å | 7.2832(2) |
| b/Å | 7.5542(2) |
| c/Å | 13.9686(4) |
| α/° | 90 |
| β/° | 98.6983(15) |
| γ/° | 90 |
| Volume/Å³ | 759.70(4) |
| Z, Z′ | 4, 2 |
| ρ_{calc} g/cm³ | 1.418 |
| μ/mm⁻¹ | 0.369 |
| F(000) | 344.0 |
| Crystal size/mm³ | 0.384 × 0.207 × 0.131 |
| Radiation | MoKα (λ = 0.71073) |
| 2Θ range for data collection/° | 2.95 to 56.582 |
| Index ranges | −9 ≤ h ≤ 9, −10 ≤ k ≤ 10, −18 ≤ l ≤ 18 |
| Reflections collected | 5810 |
| Independent reflections | 5810 [Rᵢₙₜ = 0.0580, R_{sigma} = 0.0288] |
| Data/restraints/parameters | 5810/1/186 |
| Goodness of Fit | 1.057 |
| Final R indexes [I > 2σ (I)] | R₁ = 0.0330, wR₂ = 0.0869 |
| Final R indexes [all data] | R₁ = 0.0350, wR₂ = 0.0900 |
| Δρmax, Δρmin/e Å⁻³ | 0.47/−0.53 |
| Flack Parameter | −0.02(3) |

R₁ = (Σ|Fₒ| − |F_{c}|)/Σ|Fₒ|); wR₂ = {Σ[w(Fₒ² − F_{c}²)²]/Σ[w(Fₒ²)²]}^{1/2};
S = {Σ[w(Fₒ² − F_{c}²)²]/(n − p)}^{1/2.}

Several experiments yielded diffractogram patterns that were different or, more commonly, had extra peaks observed. The extra peaks would indicate the presence of another form, albeit not in a pure phase.

Attempts to reproduce these forms failed using both crash cooling, evaporation and anti-solvent addition. Analysis of these attempts by NMR showed that the material was still predominately the NACA material and that it had not oxidized to diNACA. The lack of reproducibility of these potential forms is good evidence for their lack of stability. This study has clearly demonstrated that the NACA material exists as Form 1 and that other forms are difficult, if not impossible, to produce.

Approximately 80 mg of NACA was weighed into each of 24 vials. The solvents listed below were added to the appropriate vials. The quantities added were calculated (based on solubility studies) to dissolve approx. 60% of the material. These mixtures were temperature cycled between ambient and 40° C., in 4 hour cycles, for 72 hours. Solids isolated from the slurries are tested by XRPD. The resulting saturated solutions were separated into three separated vials for crash cooling, anti-solvent addition and evaporation experiments.

TABLE 17
List of Solvents Used
in the Primary
Polymorph Screen

| Solvent | Solvent |
|---|---|
| 1 | Acetone |
| 2 | Acetone/water (80:20) |
| 3 | Acetone/Heptane (75:25) |
| 4 | Acetonitrile |
| 5 | Acetonitrile/water (80:20) |
| 6 | 1-Butanol |
| 7 | 1,2-Dimethoxyethane |
| 8 | 1,4-Dioxane |
| 9 | Dioxane/water (80:20) |
| 10 | Ethanol |
| 11 | Ethanol/water (80:20) |
| 12 | Ethanol/heptane (75:25) |
| 13 | Ethyl Formate |
| 14 | Isopropyl acetate |
| 15 | Ethyl acetate |
| 16 | Methanol |
| 17 | Methanol/water (80:20) |
| 18 | Methyl Ethyl ketone |
| 19 | Nitromethane |
| 20 | 1-Propanol |
| 21 | 2-Propanol |
| 22 | 2-Propanol/water (80:20) |
| 23 | Tetrahydrofuran |
| 24 | Water |

Liquid Chromatography with Mass Spectrometric Detection.
Column Temperature: 30° C.
Mobile Phase A: 0.1% v/v Formic in Water
Mobile Phase B: 0.1% v/v Formic in Acetonitrile
Diluent: Mobile Phase A: 50:50 Water:Acetonitrile
Flow Rate: 1.0 mL/min
Runtime: 25 minutes
Injection Volume: 10 μL
Detection: 190-400 nm
Gradient::

| Time (minutes) | Solvent B [%] |
|---|---|
| 0 | 0 |
| 12 | 10 |
| 15 | 100 |
| 15.1 | 100 |
| 25 | 0 |

Instrument: LCQ Advantage Ion Trap MS
Sample concentration: 1 mg/ml, +ve ion mode by infusion
Source voltage (kV): 4.50
Source current (μA): 80.00
Sheath gas flow rate: 20.00
Aux/Sweep gas flow rate: 0.00
Capillary voltage (V): 8.00
Capillary temp (° C.): 200
Tube lens (V, Sp): 40.00

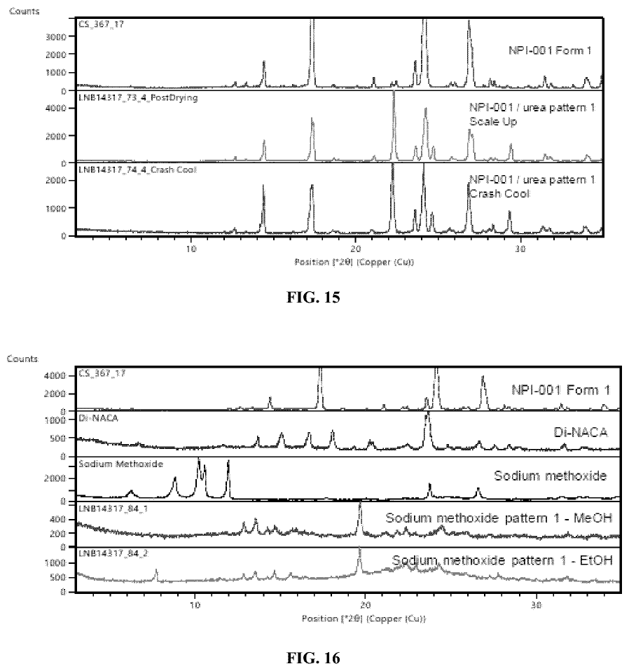

NACA/Urea Co-crystal. A primary co-crystal screen was conducted where 28 potential co-crystal formers (CCFs) were screened (Table 18) in 6 solvent systems under 2 process relevant crystallization conditions, namely, thermal maturation and evaporation. A NACA/urea co-crystal was identified (FIG. 15). The NACA/urea pattern 1 material was successfully scaled up from four solvents as a part of the secondary co-crystal screen, then fully characterized where it was found to be crystalline by XPRD and PLM with the expected XRPD pattern, thermally stable with high purity. NMR analysis confirmed an approximate stoichiometric content of urea contained within the material. The material appeared to be stable under ambient conditions and elevated temperature (80° C.) but unstable when stored at high humidity for prolonged periods, showing degradation to the diNACA. No signs of dissociation were identified in organic solvents and solvent/water mixtures with low water activity but was found to dissociate in deionized water and solvent/water mixtures with a high water activity. An additional DSC experiment with post-XRPD analysis confirmed that the exothermic event observed during the DSC cooling cycle is a recrystallization to NACA.

TABLE 18
Primary Co-Crystal
Screen Co-Former List

| | Co-Former | GRAS* |
|---|---|---|
| 1 | 2-Picolinamide | Yes |
| 2 | 5-Methylfurfural | Yes |
| 3 | 5- | Yes |
| | Methylfurfurylamine | |
| 4 | Adenine | Yes |
| 5 | Citric Acid | Yes |
| 6 | Glycine | Yes |
| 7 | Hippuric Acid | Yes |
| 8 | L-Aspartic Acid | Yes |
| 9 | L-Proline | Yes |
| 10 | L-Tyrosine | Yes |
| 11 | Malonic Acid | Yes |
| 12 | Melamine | Yes |
| 13 | Oxalic Acid | Yes |
| 14 | Theophylline | Yes |
| 15 | Tromethamine | Yes |
| 16 | Urea | Yes |
| 17 | Xanthine | Yes |
| 18 | 3,4- | Yes |
| | Dihydroxybenzoic | |
| | Acid | |
| 19 | Camphoric Acid | Yes |
| 20 | Cytosine | Yes |
| 21 | Formamide | Yes |
| 22 | L-Cysteine | Yes |
| 23 | L-Methionine | Yes |
| 24 | L-Serine | Yes |
| 25 | Threonine | Yes |
| 26 | DiNACA | Yes |
| 27 | N-Acetyl-L-cysteine | Yes |
| 28 | Succinic acid | Yes |

*GRAS: generally recognized as safe

NACA Sodium Salt. A salt screen was conducted using 6 solvent systems under 2 process-relevant crystallization conditions, namely thermal maturation and evaporation. From this study, one sodium salt was identified. The salt was formed using sodium methoxide from either acetonitrile, ethanol, methanol, or tetrahydrofuran as shown in FIG. 16.

Pharmacokinetics of NACA.

Human plasma concentrations of N-acetylcysteine amide (NACA) and its major metabolite, N-acetylcysteine (NAC), upon dosing NACA Tablets, 250 mg, once daily for 1-2 months, then twice daily (BID) (500 mg/day total dose), for up to 9 months, are shown in FIG. 17. Human plasma concentrations of NACA after last dose of NACA oral solution, twice daily, for 14 days, are shown in FIG. 18. Human plasma concentrations of NAC after last dose of NACA oral solution, twice daily, for 14 days, are shown in FIG. 19. The maximum plasma concentration (Cmax) of NACA after the last 500 mg BID oral solution dose on day 14, i.e., was about 900 ng/ml (after 1 g/day total oral dose). The mean plasma concentrations of NACA after 3-9 months of NACA Tablets, 250 mg, BID dosing (i.e., 500 mg/day), was about 600-800 ng/ml (after 0.5 g/day oral dose), greater than expected compared to the plasma concentrations of the oral NACA solution. However, the pharmacokinetic profile of the oral NACA solution was accurately timed after last dose whereas the profile of the tablet was not accurately timed after the most recent dose.

### Example 1

The dry blend for NACA tablets (Table 11) was roller compacted at 200 pounds per square inch (PSI) and milled. The first portion of the milled blend was transferred to the compression machine fitted with 11 mm diameter punches. Poor powder flow was observed and 2 out of 10 compressed tablets failed to meet target weight and thickness ranges (Table 19). Even after the roller compaction force was increased to 300 PSI, 2 out of 10 tablets failed to meet target weight and thickness ranges. The remaining blend was compressed into tablets, then milled and compressed into tablets again. This pharmaceutical technique of dry granulation is called “slugging” (Sun and Kleinebudde, 2016), and obviates roller compaction. As a surprising result of trial and error with “slugging,” (a) powder flow was much improved and (b) all tablets met target ranges for weight and thickness.

TABLE 19
Slugging Dry Granulation Process Improved Weight and Thickness of NACA Tablets

| | | | | | Tablet | Tablet | |
|---|---|---|---|---|---|---|---|
| | | | | | weight | Thickness | |
| | | | | | (Target: | (Target: | |
| | Tablet | Disintegration | Friability | | 475.0 to | 5.10 to | |
| Dry Granulation | hardness | time (target | (NMT | Powder | 525.0 | 5.30 | |
| Techniques | (avg) | NMT 10 min) | 1.0%) | Flow | mg)* | mm)* | Result** |
| Roller compaction @ | 9.3 kp | 43-55 sec | 0.26% | Poor | 450 to | 4.96 to | 00S |
| 200 PSI followed | | | | | 550 mg | 5.5 mm | |
| by compression | | | | | | | |
| Roller compaction @ | 6 kp | NT | NT | Poor | 494.1 to | 5.10 to | 00S |
| 300 PSI followed | | | | | 531.8 | 5.38 mm | |
| by compression | | | | | mg | | |
| ″Slugging″ | 6 kp | 49-55 sec | 0.41% | Improved | 487.9 to | 5.10 to | Pass |
| followed by | | | | | 513.2 | 5.25 mm | |
| compression | | | | | mg | | |

*In-process controls specifications; **OOS: Out of specification for tablet weight and thickness; NMT: Not more than; min: minutes; avg: average; NT: Not tested.

However, the skilled artisan will understand that the first dry blend can be compacted at 50, 60, 70, 75, 80, 90, 100, 110, 120, 125, 130, 140, 150, 160, 170, 175, 180, 190, 200, 225, 250, 275, 300 or more pounds per square inch (PSI) and milled. The first dry blend can then be compacted at 50, 60, 70, 75, 80, 90, 100, 110, 120, 125, 130, 140, 150, 160, 170, 175, 180, 190, 200, 225, 250, 275, 300, 350, 400, 450, 500, or more pounds per square inch (PSI) and then again compacted.

As used herein, a tablet that is in specification (meets specification) for tablet weight and thickness is a tablet that meets the in-process control testing target ranges for tablet weight and thickness listed in Table 19. When a tablet fails to meet the in-process requirements of for tablet weight and thickness, then the tablet is said to be out of specification. Release specifications for NACA tablets are listed in Table 20.

TABLE 20
Release Specifications for NACA Tablets.

| Test | Method | Acceptance Criteria |
|---|---|---|
| Appearance | Visual | White to off-white, round, |
| | Assessment | biconvex tablets |
| Identification | HPLC | Retention time and UV spectrum |
| | | conforms to reference standard |
| Related Substances | HPLC | Report any impurity ≥ 0.05% by |
| | | RRT and % |
| Assay | HPLC | 95.0%-105.0% label claim |
| Chiral Purity | HPLC | D-NACA ≤ 0.2% |
| Dissolution | HPLC | Report results |
| Content Uniformity | HPLC | Complies with USP <905> |
| Water Content | USP <921>, | Report results |
| | 1c | |
| Total Aerobic | USP <61> | NMT 1000 CFU/g |
| Microbial Count | | |
| Total Combined | USP <61> | NMT 100 CFU/g |
| Mold and Yeast | | |
| Count | | |
| | USP <62> | Absence of Escherichia coli in 1 g |

### Embodiments

Embodiment 1. A tablet comprising N-acetylcysteine amide (NACA) and a NACA-acceptable, biocompatible excipient and optionally one or more pharmaceutically acceptable additives, binders, or fillers.

Embodiment 2. The tablet of embodiment 1, wherein the one or more pharmaceutically acceptable additives, binders, or fillers are selected from microcrystalline cellulose, lactose monohydrate, croscarmellose sodium and stearic acid.

Embodiment 3. The tablet of embodiments 1 or 2, wherein the NACA comprises from 1, 2, 3, 4, 5, 6, 7, 8, 9, 10, 15, 20, 25, 30, 35, 40, 45, 50, 55, 60, 70, or 75 weight percent (wt. %) of the tablet.

Embodiment 4. The tablet of any one of embodiments 1 to 3, wherein the NACA comprises 10, 20, 30, 40, 50, 60, or 70 wt. % of the tablet.

Embodiment 5. The tablet of any one of embodiments 1 to 4, wherein the tablet is 100, 200, 250, 300, 350, 400, 450, 500, 600, 700, 750, 800, 900, 1,000, 1,250, 1,500, 1,750, or 2,000 mg.

Embodiment 6. The tablet of any one of embodiments 2 to 5, wherein the lactose is 8.575 wt. %, but can range from 0, 1, 2, 3, 4, 5, 6, 7, 8, 9 10, 15, 20, 25, 30, 35, 40, 45, 50, 55, 60 to 66% with corresponding adjustments to other components.

Embodiment 7. The tablet of any one of embodiments 2 to 6, wherein the microcrystalline cellulose is 34.425 wt. %, but can range from 0, 1, 2, 3, 4, 5, 6, 7, 8, 9 10, 15, 20, 25, 30, 35, 40, 45, 50, 55, 60, 70, 75, 80, 85, 90 to 91% with corresponding adjustments to other components.

Embodiment 8. The tablet of any one of embodiments 2 to 7, wherein the croscarmellose sodium is 5.0%, but can range from 0, 1, 2, 3, 4, 5, 6, 7, 8 to 9% with corresponding adjustments to other components.

Embodiment 9. The tablet of any one of embodiments 2 to 8, wherein the stearic acid is 1.0 wt. %, but can range from 0.1, 0.2, 0.3, 0.4, 0.5, 0.6, 0.7, 0.8, 0.9, 1, 2, 3, 4, 5 to 5.6% with corresponding adjustments to other components.

Embodiment 10. The tablet of any one of embodiments 2 to 9, wherein the tablet comprises an NACA-acceptable, biocompatible excipient and one or more pharmaceutically acceptable additives, binders, or fillers.

Embodiment 11. The tablet of any one of embodiments 2 to 10, wherein the one or more pharmaceutically acceptable additives, binders, or fillers are selected from microcrystalline cellulose, lactose monohydrate, croscarmellose sodium and stearic acid.

Embodiment 12. The tablet of any one of embodiments 2 to 11, wherein the tablets are dry granulated by roller compaction.

Embodiment 13. The tablet of any one of embodiments 2 to 12, wherein the tablets are dry granulated by slugging.

Embodiment 14. The tablet of any one of embodiments 2 to 13, wherein the NACA tablet weight is in the range of approximately 475 to 525 mg.

Embodiment 15. The tablet of any one of embodiments 2 to 14, wherein the NACA tablet thickness is in the range of approximately 4 to 6 mm, 4.5 to 5.5 mm or 5 mm.

Embodiment 16. The tablet of any one of embodiments 2 to 15, wherein the NACA tablet width is in the range of approximately 10 to 12 mm or 11 mm.

Embodiment 17. The tablet of any one of embodiments 2 to 16, wherein the NACA tablet hardness is in the range of approximately 3 to 12 kp or 6 kp.

Embodiment 18. The tablet of any one of embodiments 2 to 17, wherein the NACA tablet disintegration time is less than 20, 15, 10 or 5 minutes or approximately less than 1 minute.

Embodiment 19. The tablet of any one of embodiments 2 to 18, wherein the NACA tablet friability is not more than 5, 4, 3, 2 or 1.0% or approximately less than 1%.

Embodiment 20. The tablet of any one of embodiments 2 to 19, wherein the NACA tablet tablets content uniformity complies to meets or exceeds US Pharmacopeia (USP) 905.

Embodiment 21. The tablet of any one of embodiments 2 to 20, wherein the NACA tablet is a white to off-white, round, biconvex tablet.

Embodiment 22. The tablet of any one of embodiments 2 to 21, NACA tablet is a coated or colored tablet. In another aspect, the NACA tablet has an elliptical or oval shape.

Embodiment 23. A method of making a pharmaceutical composition in the form an oral tablet comprising N-acetylcysteine amide (NACA), comprising: compressing the NACA and the NACA-acceptable, biocompatible excipient to form first tablets, crushing or milling the first tablets, and compressing the crushed or milled first tablets into slugged tablets, wherein the slugged tablets have greater than 90, 91, 92, 93, 94, 95, 96, 97, 98, or 100% of the slugged tablets meet specification, wherein the composition has a pharmacokinetic (PK) profile comprising mean plasma concentrations of NACA and NAC ranging from about 200 to 1200 ng/ml after administration of the pharmaceutical composition to a human.

Embodiment 24. The method of embodiment of 23, wherein slugging the tablet comprises compressing the crushed or milled first tablets into slugged tablets, wherein the slugged tablets have greater than 90, 91, 92, 93, 94, 95, 96, 97, 98, or 100% of the slugged tablets meet specification.

Embodiment 25. The method of embodiments 23 or 24, wherein the NACA tablet hardness is in the range of approximately 3 to 12 kp or 6 kp.

Embodiment 26. The method of any one of embodiments 23 to 25, wherein the NACA tablet disintegration time is less than 20, 15, 10 or 5 minutes or approximately less than 1 minute.

Embodiment 27. The method of any one of embodiments 23 to 26, wherein the NACA tablet friability is not more than 5,4, 3, 2 or 1.0% or approximately less than 1%.

Embodiment 27. The method of any one of embodiments 23 to 26, wherein the NACA tablet tablets content uniformity complies to meets or exceeds US Pharmacopeia (USP) 905.

Embodiment 28. The method of any one of embodiments 23 to 27, wherein the NACA tablet is a white to off-white, round, biconvex tablet.

Embodiment 29. The method of any one of embodiments 23 to 28, NACA tablet is a coated or colored tablet. In another aspect, the NACA tablet has an elliptical or oval shape.

Embodiment 30. A capsule comprising N-acetylcysteine amide (NACA) and a NACA-acceptable, biocompatible excipient and optionally one or more pharmaceutically acceptable additives, binders, or fillers.

It is contemplated that any aspects of the disclosure discussed in this specification can be implemented with respect to any method, kit, reagent, or composition of the disclosure, and vice versa. Furthermore, compositions of the disclosure can be used to achieve methods of the disclosure.

It will be understood that particular aspects described herein are shown by way of illustration and not as limitations of the disclosure. The principal features of this disclosure can be employed in various aspects without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In aspects of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed disclosure. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the disclosure(s) set out in any claims that may issue from this disclosure. Specifically, and by way of example, although the headings refer to a “Technical Field,” such claims should not be limited by the language under this heading to describe the so-called technical field. Further, a description of technology in the “Background” section is not to be construed as an admission that technology is prior art to any disclosure(s) in this disclosure. Neither is the “Summary” to be considered a characterization of the disclosure(s) set forth in issued claims. Furthermore, any reference in this disclosure to “invention” in the singular should not be used to argue that there is only a single point of novelty in this disclosure. Multiple aspects may be set forth according to the limitations of the multiple claims issuing from this disclosure, and such claims accordingly define the aspect(s), and their equivalents, that are protected thereby. In all instances, the scope of such claims shall be considered on their own merits in light of this disclosure but should not be constrained by the headings set forth herein.

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A tablet comprising N-acetylcysteine amide (NACA) and a NACA-acceptable, biocompatible excipient and optionally one or more pharmaceutically acceptable additives, binders, or fillers.

2. The tablet of claim of 1, wherein the one or more pharmaceutically acceptable additives, binders, or fillers are selected from microcrystalline cellulose, lactose monohydrate, croscarmellose sodium and stearic acid.

3. The tablet of claim of 1, wherein the NACA comprises from 1, 2, 3, 4, 5, 6, 7, 8, 9, 10, 15, 20, 25, 30, 35, 40, 45, 50, 55, 60, 70 to 75 weight percent (wt. %) of the tablet.

4. The tablet of claim of 1, wherein the NACA comprises 10, 20, 30, 40, 50, 60, or 70 wt. % of the tablet.

5. The tablet of claim of 1, wherein the tablet is 100, 200, 250, 300, 350, 400, 450, 500, 600, 700, 750, 800, 900, 1,000, 1,250, 1,500, 1,750, or 2,000 mg.

6. The tablet of claim of 2, wherein the lactose is 8.575 wt. %, but can range from 0, 1, 2, 3, 4, 5, 6, 7, 8, 9 10, 15, 20, 25, 30, 35, 40, 45, 50, 55, 60 to 66 wt. % with corresponding adjustments to other components.

7. The tablet of claim of 2, wherein the microcrystalline cellulose is 34.425 wt. %, but can range from 0, 1, 2, 3, 4, 5, 6, 7, 8, 9 10, 15, 20, 25, 30, 35, 40, 45, 50, 55, 60, 70, 75, 80, 85, 90 to 91 wt. % with corresponding adjustments to other components.

8. The tablet of claim of 2, wherein the croscarmellose sodium is 5.0 wt. %, but can range from 0, 1, 2, 3, 4, 5, 6, 7, 8 to 9 wt. % with corresponding adjustments to other components.

9. The tablet of claim of 2, wherein the stearic acid is 1.0 wt. %, but can range from 0.1, 0.2, 0.3, 0.4, 0.5, 0.6, 0.7, 0.8, 0.9, 1, 2, 3, 4, 5 to 5.6 wt. % with corresponding adjustments to other components.

10. The tablet of claim of 1, wherein the tablets are dry granulated by roller compaction.

11. The tablet of claim of 1, wherein the tablets are dry granulated by slugging.

12. The tablet of claim of 1, wherein the tablet weight is in the range of approximately 475 to 525 mg.

13. The tablet of claim of 1, wherein the tablet thickness is in the range of approximately 4 to 6 mm, 4.5 to 5.5 mm, or about 5 mm.

14. The tablet of claim of 1, wherein the tablet width is in the range of approximately 10 to 12 mm, or about 11 mm.

15. The tablet of claim of 1, wherein the tablet hardness is in the range of approximately 3 to 12 kp, or about 6 kp.

16. The tablet of claim of 1, wherein the tablet disintegration time is less than 20, 15, 10 or 5 minutes, or approximately less than 1 minute.

17. The tablet of claim of 1, wherein the tablet friability is not more than 5, 4, 3, 2 or 1.0%, or approximately less than 1%.

18. The tablet of claim of 1, wherein the tablets content uniformity complies to US Pharmacopeia (USP) 905.

19. The tablet of claim of 1, wherein the tablet comprises D-NACA≤0.2%.

20. The tablet of claim of 1, wherein the tablet is a white to off-white, round, biconvex tablet.

21. The tablet of claim of 1, wherein the tablet is a coated or colored tablet.

22. The tablet of claim of 1, wherein the tablet has an elliptical or oval shape.

23. A method of making a pharmaceutical composition in the form an oral tablet comprising N-acetylcysteine amide (NACA), comprising:
compressing the NACA and the NACA-acceptable, biocompatible excipient to form first tablets,
crushing or milling the first tablets, and
compressing the crushed or milled first tablets into slugged tablets, wherein the slugged tablets have greater than 90, 91, 92, 93, 94, 95, 96, 97, 98, or 100% of the slugged tablets meet one or more specifications as set forth in Table 20, wherein the pharmaceutical composition has a pharmacokinetic (PK) profile comprising mean plasma concentrations of NACA and N-acetylcysteine (NAC) ranging from about 200 to 1200 ng/ml after administration of the pharmaceutical composition to a human.

24. The method of claim of 23, wherein slugging the tablet comprises compressing the crushed or milled first tablets into slugged tablets, wherein the slugged tablets have greater than 90, 91, 92, 93, 94, 95, 96, 97, 98, or 100% of the slugged tablets meet at least one of assay, target weight, thickness range, content uniformity, or chiral purity specification.

25. The method of claim of 23, wherein the tablet hardness is in the range of approximately 3 to 12 kp, or about 6 kp.

26. The method of claim of 23, wherein the tablet disintegration time is less than 20, 15, 10 or 5 minutes, or approximately less than 1 minute.

27. The method of claim of 23, wherein the tablet friability is not more than 5, 4, 3, 2, 1.0%, or approximately less than 1%.

28. The method of claim of 23, wherein the tablet tablets content uniformity complies to US Pharmacopeia (USP) 905.

29. The method of claim of 23, wherein the tablet comprises D-NACA≤0.2%.

30. The method of claim of 23, wherein the tablet is a white to off-white, round, biconvex tablet.

31. The method of claim of 23, wherein the tablet is a coated or colored tablet.

32. The method of claim of 23, wherein the tablet has an elliptical or oval shape.

33. A capsule comprising N-acetylcysteine amide (NACA) and a NACA-acceptable, biocompatible excipient and optionally one or more pharmaceutically acceptable additives, binders, or fillers.
